(19) United States
(12) Patent Application Publication
(10) Pub. No.: US 2007/0059286 A1
(43) Pub. Date: Mar. 15, 2007

(54) Dna vaccines that express an adp-ribosyltransferase toxin devoid of adp-ribosyltransferase activity

(76) Inventor: David M. Hone, Poolesville, MD (US)

Correspondence Address: INTELLECTUAL PROPERTY / TECHNOLOGY LAW, PO BOX 14329, RESEARCH TRIANGLE PARK, NC, 27709, US

(21) Appl. No.: 10/545,517

(22) PCT Filed: Sep. 2, 2003
(86) PCT No.: PCT/US03/27479

§ 371 Date: Oct. 5, 2006

Related U.S. Application Data
(60) Provisional application No. 60/447,460, filed on Feb. 14, 2003.

(51) Int. Cl. A61K 48/00; C12N 15/74; A61K 39/02; C12N 1/21; A61K 39/106

(52) U.S. Cl. 424/93.2; 435/252.3; 435/471; 424/200.1; 514/44; 424/261.1

(57) ABSTRACT

The present invention describes DNA vaccines that encode for and direct the coincident expression of an antigen and an ADP-ribosyltransferase toxin that is devoid of ADPribosyltransferase activity and methods for vaccinating animals with the same. The DNA vaccines are useful for vaccinating against viral, bacterial and parasitic pathogens.

### BACKGROUND OF THE INVENTION

1. Field of the Invention

The present invention relates to DNA vaccines, and more particularly to DNA vaccines that direct the coincident expression of an antigen and an ADP-ribosyltransferase toxin devoid of ADP-ribosyltransferase activity, and methods for vaccinating animals with the same.

2. Background of the Related Art

The prior art pertinent to the current invention describes a diverse array of conventional DNA vaccines, which are generally comprised of a plasmid vector, a promoter for transcription initiation that is active in eukaryotic cells, and a vaccine antigen (Gurunathan et al., Ann. Rev. Immunol., 18:927 (2000); Krieg, Biochim. Biophys. Acta., 1489:107 (1999); Cichutek, Dev. Biol. Stand., 100:119 (1999); Davis, Microbes Infect., 1:7 (1999); Leitner, Vaccine, 18:765 (1999)).

Examples of plasmid vectors that have been used in conventional DNA vaccines include pBR322 (ATCC# 31344); pUC19 (ATCC# 37254); pcDNA3.1 (Invitrogen, Carlsbad Calif. 92008; Cat. No. V385-20; DNA sequence available at http://www.invitrogen.com/vectordata/index.html); pNGVL (National Gene Vector Laboratory, University of Michigan, Mich.); p414cyc (ATCC# 87380), p414GALS (ATCC# 87344), pBAD18 (ATCC# 87393), pBLCAT5 (ATCC# 77412), pBluescriptIIKS, (ATCC# 87047), pBSL130 (ATCC# 87145), pCM182 (ATCC# 87656), pCMVtkLUC (ATCC# 87633), pECV25 (ATCC#77187), pGEM-7zf (ATCC# 87048), pGEX-KN (ATCC# 77332), pJC20 (ATCC# 87113, pUB110 (ATCC# 37015), pUB18 (ATCC# 37253).

Examples of promoters that have been used in conventional DNA vaccines include the SV40 early promoter (GenBank accession # M99358, Fiers et al. Nature, 273: 113-120 (1978)), the cytomegalovirus immediate early promoter/enhancer (Genebank accession # AF025843) and the rous sarcoma virus long terminal repeat (Genebank accession # M83237; Lon et al. Hum. Immunol., 31: 229-235 (1991)) promoters, or the eukaryotic promoters or parts thereof, such as the β-casein (Genebank accession # AF194986; ref Fan et al. Direct submission (2000)), uteroglobin (Genebank accession # NM003357; ref Hay et al. Am. J. Physiol., 268: 565-575 (1995)), β-actin (Genebank accession # NM001101; ref Vandekerckhove and Weber. Proc. Natl. Acad. Sci. U.S.A., 73: 1106-1110 (1978)), ubiquitin (Genebank accession # AJ243268; Robinson. Direct Submission, (2000)) or tyrosinase (Genebank accession # NM000372; Shibaharo et al. Tohoku J. Exp. Med., 156: 403-414 (1988)) promoters.

Examples of vaccine antigens that have been used in conventional DNA vaccines include Plasniodium vivax and Plasmodium falciparum antigens; Entamoeba histolytica antigens Hepatitis C virus antigens, Hepatitis B virus antigens, HIV-1 antigens, Semliki Forest virus antigens, Herpes Simplex viral antigens, Pox virus antigens, Influenza virus antigens, Measles virus antigens, Dengue virus antigens, Papilloma virus antigens (A comprehensive reference database of DNA vaccine citations can be obtained from URL:http://www.DNAvaccine.com/Biblio/articles.html).

Since their inception in 1993, conventional DNA vaccines encoding an antigen under the control of a eukaryotic or viral promoters have been used to immunize rodents (e.g. mice, rats and guinea pigs), swine, chickens, ferrets, non-human primates and adult volunteers (Webster et al, Vacc., 12:1495-1498 (1994); Bernstein et al., Vaccine, 17:1964 (1999); Huang et al., Viral Immunol., 12:1 (1999); Tsukamoto et al., Virology, 257:352 (1999); Sakaguchi et al., Vaccine, 14:747 (1996); Kodihalli et al., J. Virol., 71: 3391 (1997); Donnelly et al., Vaccine, 15:865 (1997); Fuller et al., Vaccine, 15:924 (1997); Fuller et al., Immunol. Cell Biol., 75: 389 (1997); Le et al., Vaccine, 18:1893 (2000); Boyer et al., J. Infect. Dis., 181:476 (2000)).

Although conventional DNA vaccines induce immune responses against a diverse array of antigens, the magnitudes of the immune responses have not always been sufficient to engender protective immunity. Several approaches have been developed to increase the immunogenicity of conventional DNA vaccines, including the use of altered DNA sequences, such as the use of antigen-encoding DNA sequences optimized for expression in mammalian cells (Andre, J. Virol., 72:1497 (1998); Haas, et al., Curr. Biol. 6:315-24 (1996); zur Megede, et al., J. Virol., 74:2628 (2000); Vinner, et al., Vaccine, 17:2166 (1999)) or incorporation of bacterial immunostimulatory DNA sequence motifs (i.e. the CpG motif) (Krieg, Biochim. Biophys. Acta., 1489:107 (1999); McAdam et al. J. Virol., 74: 203-208 (2000); Davis, Curr. Top. Microbiol. Immunol., 247:17 (2000); McCluskie, Crit. Rev. Immunol., 19:303 (1999); Davis, Curr. Opin. Biotechnol., 8:635 (1997); Lobell, J. Immunol., 163:4754 (1999)). The immunogenicity of conventional DNA vaccines can also be modified by formulating the conventional DNA vaccine with an adjuvant, such as aluminum phosphate or aluminum hydroxyphosphate (Ulmer et al., Vaccine, 18:18 (2000)), monophosphoryl-lipid A (also referred to as MPL or MPLA; Schneerson et al. J. Immunol., 147: 2136-2140 (1991); Sasaki et al. Inf. Immunol., 65: 3520-3528 (1997); Lodmell et al. Vaccine, 18: 1059-1066 (2000)), QS-21 saponin (Sasali, et al., J. Virol., 72:4931 (1998); dexamethasone (Malone, et al., J. Biol. Chem. 269:29903 (1994); CpG DNA sequences (Davis et al., J. Immunol., 15:870 (1998); a cytoline (Hayashi et al. Vaccine, 18: 3097-3105 (2000); Sin et al. J. Immunol., 162: 2912-2921 (1999); Gabaglia et al. J. Immunol., 162: 753-760 (1999); Kim et al., Eur J Immunol., 28:1089 (1998); Kim et al., Eur. J. Immunol., 28:1089 (1998); Barouch et al., J. Immunol., 161:1875 (1998); Okada et al., J. Immunol., 159:3638 (1997); Kim et al., J. Virol., 74:3427 (2000)), or a chemoline (Boyer et al., Vaccine 17(Suppl 2):S53 (1999); Xin et al., Clin. Immunol., 92:90 (1999)). In each of the above cited instances the immunogenicity of the conventional DNA vaccines was enhanced or modified, thus validating the idea that the immunogenicity of conventional DNA vaccines can be influenced through the use of adjuvant.

Cholera toxin (Ctx) is a well-known adjuvant that is typically used to augment the immunogenicity of mucosal vaccines, such as those given intranasally or orally (Xu-Amano, et al., J. Exp. Med., 178:1309 (1993); VanCott, et al., Vaccine, 14:392 (1996); Jackson, R. J. et al., Infect. Immun., 61:4272 (1993); Marinaro, M. et al., Ann. New York Acad. Sci., 795:361 (1996); Yamamoto, S. et al. J. Exp. Med. 185:1203 (1997); Porgador, et al., J. Immunol., 158:834 (1997); Lycke and Holmgren, Monogr., Allergy, 24:274 (1988); Homquist and Lycke, Eur. J. Immunol. 23:2136 (1993); Hornquist, et al., Immunol., 87:220 (1996); Agren, et al., Immunol. Cell Biol., 76:280 (1998)).

The adjuvant activity of Ctx is mediated by the A1 domain of the A subunit of Ctx (herein referred to as CtxA1); chimeric proteins comprised of an antigen fused to CtxA1 demonstrate that CtxA1 alone possesses adjuvant activity (Agren, et al., J. Immunol., 164:6276 (2000); Agren, et al., Immunol, Cell Biol., 76:280 (1998); Agren, et al., J. Immunol., 158:3936 (1997)). The utilization of the A subunit, the A1 domain of Ctx or analogues thereof in a DNA vaccine has not heretofore been reported. More recently the use of Ctx as an adjuvant has been extended to transcutaneous vaccines (Glenn et al., Infect. Immun., 67:1100 (1999); Scharton-Kersten et al., Vaccine 17(Suppl. 2):S37 (1999)). Thus, recent evidence suggests that cholera toxin (CT) as an adjuvant applied topically with an antigen to the skin surface (i.e. transcutaneous vaccination) elicits IgG responses against the antigen, whereas topical application of the antigen alone does not induce detectable IgG response (Glenn et al., supra (1999); Scharton-Kersten et al., supra (1999)). Since Ctx is a member of the family of bacterial adenosine diphosphate-ribosylating exotoxins, other members of this family, E.g. the heat-labile toxins (Herein referred to as Ltx) of enterotoxigenic Escherichia coli, also possess adjuvant activity (Rappuoli et al., Immunol. Today, 20:493 (1999)).

The idea that ADP-ribosyltransferase activity was required for the adjuvanticity of both CT and LT stemmed from studies with CTB and mutant derivatives of CT and LT in which the serine in position 63 of the mature A1 subunit have been replaced by lysine (referred to herein as “CT-K63” and “LT-K63”, respectively). All of these toxin derivatives, i.e. CTB, CT-K63 and LT-K63, lack ADP-ribosyltransferase activity and are relatively ineffective as adjuvants (Stevens, et al., Infect Immun 67:259-265 (1999); Yamamoto, et al., J. Exp. Med. 185:1203-1210 (1997); Douce, et al. Infect Immun 67:4400-4406 (1999)). In contrast, mutants of CT and LT that display diminished ADP-ribosyltransferase activity are still modestly effective as adjuvants (Stevens, et al., Infect Immun 67:259-265 (1999); Yamamoto, et al., J. Exp. Med. 185:1203-1210 (1997); Douce, et al. Infect Immun 67:4400-4406 (1999)). Thus, the prior art teaches that the potent adjuvanticity of CT and LT and mutant derivatives thereof requires some intrinsic ADP-ribosyltransferase activity.

### SUMMARY OF THE INVENTION

In one aspect the present invention relates to DNA vaccines that direct the coincident expression of an antigen and an ADP-ribosyltransferase toxin devoid of ADP-ribosyltransferase activity. The vaccines are useful for vaccinating against viral, bacterial, arasitic pathogens, autoimmune antigens and transplantation antigens.

In another aspect, the present invention relates to novel compositions and methods of use as DNA vaccines that express of ADP-ribosyltransferase toxins that are deficient in intrinsic ADP-ribosyltransferase activity and yet retain adjuvanticity.

The DNA vaccines of the present invention that express an adjuvant devoid in ADP-ribosyltransferase activity significantly augments immune responses to vaccine antigens encoded on the specific DNA vaccine. Moreover, DNA vaccines that express an adjuvant devoid in ADP-ribosyltransferase activity are not hampered by the safety concerns relative to those applicable in DNA vaccines that express an adjuvant exhibiting ADP-ribosyltransferase activity.

Heretofore, there is no documentation showing that CT and LT derivatives devoid of ADP-ribosyltransferase activity are adjuvant. That is, the present invention provides the first documentation that DNA vaccines that direct the coexpression of an antigen and CT or LT derivatives devoid of ADP-ribosyltransferase activity are more effective than the DNA vaccine that only expresses the antigen alone.

Another aspect of the present invention relates to DNA construct that direct the coexpression of an antigen and CT or LT derivatives devoid of ADP-ribosyltransferase activity.

Yet another object of the invention is to provide DNA vaccines comprising a nucleotide sequence encoding for an antigen and CT or LT derivatives devoid of ADP-ribosyltransferase activity, and that can be used as prophylactic or therapeutic vaccines.

A still further aspect of the present invention relates to a method for enhancing the efficacy of a vaccine in a subject. The method generally comprises administering to the subject: a DNA vaccine comprising (i) a nucleic acid encoding an antigen against which an immune response is desired in the subject; and (ii) a nucleic acid encoding a mutated A1 domain of the A subunit of CT to inhibit ADP-ribosyltransferase activity. The first component and second component are administered in an immunizingly effective amount (as defined herein). In the method aspect of the invention, the first component and the second component are provided as nucleic acid sequences on the same or on separate nucleic acids and are administered directly to the subject. The first component and the second component may also be provided as nucleic acid sequences on the same or on separate nucleic acids and may be used to transform a cell, which cell is administered to the subject.

The nucleic acid sequences are preferably expressed in a coordinated and co-expressed manner upon introduction into a subject to produce an amount of the first component that is immunogenic and an amount of the second component that is effective to enhance the efficacy of the vaccine.

A related aspect of the invention involves the administration of this nucleic acid to a subject in need thereof to elicit an immune response to the antigen. The DNA vaccine comprising the at least two sequences is suitably administered as a component of a pharmaceutical composition and may be administered directly to the subject and/or introduced into a suitable host cell and said suitable host cell is administered to the subject. The host cell may be obtained from the subject or from a cell culture originating from one or more cells obtained from the subject.

In another aspect, the invention relates to a method for improving the speed of an antibody response to a soluble antigen in a subject, comprising administering to the subject the DNA vaccines of the present invention. The subject is preferably a human.

The invention also relates to compositions for achieving the various method aspects of the invention. For example, in one aspect, the invention relates to a composition comprising a first component selected from the group consisting of: (i) an antigen against which an immune response is desired in the subject, and (ii) a nucleic acid encoding the antigen of (i); along with a second component selected from the group consisting of: (i) a bacterial adenosine diphosphate-ribosylating exotoxin mutated to inhibit ADP-ribosyltransferase activity, and (ii) a nucleic acid encoding the exotoxin of (i). This composition preferably also comprises one or more of each of the following pharmaceutically acceptable components: carriers; excipients; auxiliary substances; adjuvants; wetting agents; emulsifying agents; pH buffering agents; and other components known for use in vaccine or other pharmaceutical compositions.

Yet another aspect relates to an isolated and purified polynucleotide that encodes an antigen and ADP-ribosyltransferase toxins that are devoid of ADP-ribosyltransferase activity. In a preferred embodiment, the polynucleotide of the present invention is a DNA molecule.

These and other aspects of the present invention, which will be apparent from the detailed description of the invention provided hereinafter, have been met by providing DNA vaccines that direct the coexpression of an antigen and derivative of an ADP-ribosyltransferase toxin that is devoid of ADP-ribosyltransferase activity and that retains potent adjuvanticity.

### BRIEF DESCRIPTION OF THE FIGURES

FIG. 1 shows the expression cassettes of the various DNA vaccines configurations described in the examples. In each instance the expression cassettes are located in expression vectors pcDNA3.1ZEO or pRc/CMV, which place expression under the control of the CMV promoter (PCMv).

FIG. 2 shows the expression cassettes of the DNA vaccines configurations that utilize two eukaryotic promoters (i.e. P₁ and P₂).

FIG. 3 shows the serum IgG responses to gp120 in mice 28 weeks after vaccination.

FIG. 4 shows the mutant CT-K63 holotoxin when added in the form of a purified protein must traffic via the golgi apparatus to reach the cell cytoplasm and during this transport is exposed to the cellular ubiquitination/proteosome degradation machinery. The presence of the surface-exposed lysine (i.e. K63) serves as a cognate recognition motif for ubiquitination and proteosome degradation.

FIG. 5 shows a possible mechanism through which CtxA1-K63 DNA vaccine retains adjuvant activity by a conformational change following the interaction between CtxA1-K63 and the host ARF, thereby opening the NAD-binding cleft in said mutant toxin.

FIG. 6 shows an alternative mechanism through which CtxA1-K63 DNA vaccine retains adjuvant activity by the binding of CtxA1 to the ADP-ribosyltransferase factor (ARF) to stimulate the GTPase activity of ARF; the activated ARF may then produce a signal that results in differentiation of dendritic cells that harbor the DNA vaccine into a mature antigen presenting cell, which in turn promote the profound humoral responses to the DNA vaccine-encoded immunogen.

### DETAILED DESCRIPTION OF THE PREFERRED EMBODIMENT

The term “pharmaceutically acceptable” as used herein refers to a component (such as a salt, carrier, excipient or diluent) of a formulation according to the present invention is a component which (1) is compatible with the other ingredients of the formulation in that it can be combined with the active ingredients (e.g. chemokine and/or antigen) of the invention without eliminating the biological activity of the active ingredients; and (2) is suitable for use in animals (including humans) without undue adverse side effects (such as toxicity, irritation, and allergic response). Side effects are “undue” when their risk outweighs the benefit provided by the pharmaceutical composition.

The term “immunizingly effective” is used herein refers to an immune response which confers immunological cellular memory upon the subject, with the effect that a secondary response (to the same or a similar antigen) is characterized by one or more of the following characteristics: shorter lag phase in comparison to the lag phase resulting from a corresponding exposure in the absence of immunization; production of antibody which continues for a longer period than production of antibody for a corresponding exposure in the absence of such immunization; a change in the type and quality of antibody produced in comparison to the type and quality of antibody produced from such an exposure in the absence of immunization; a shift in class response, with IgG antibodies appearing in higher concentrations and with greater persistence than IgM; an increased average affinity (binding constant) of the antibodies for the antigen in comparison with the average affinity of antibodies for the antigen from such an exposure in the absence of immunization; and/or other characteristics known in the art to characterize a secondary immune response.

The terms “peptide,” “polypeptide,” and “protein” are used interchangeably herein and are intended to refer to amino acid sequences of any length.

The term “transfected” as used herein refers to cells that have incorporated the delivered foreign DNA vaccine, whichever delivery technique is used.

The term “DNA vaccines” as used herein refers to a DNA that is introduced into cell tissue and therein expressed by cells to produce a messenger ribonucleic acid (mRNA) molecule, which is translated to produce an antigen protein and a mutated ADP-ribosyltransferase toxin that is devoid of ADP-ribosyltransferase activity

The term “foreign antigen” as used herein refers to a protein or fragment thereof, which is foreign to the recipient animal cell or tissue, such as, but not limited to, a viral protein, a parasite protein, an immunoregulatory agent, or a therapeutic agent.

The term “endogenous antigen” as used herein refers to a protein or part thereof that is naturally present in the recipient animal cell or tissue, such as, but not limited to, a cellular protein, a immunoregulatory agent, or a therapeutic agent.

The term “nucleotide variant” as used herein refers to a sequence that differs from the recited nucleotide sequence in having one or more nucleotide deletions, substitutions or additions. Such modifications may be readily introduced using standard mutagenesis techniques, such as oligonucleotide-directed site-specific mutagenesis as taught, for example, by Adelman et al. (DNA, 2:183, 1983). Nucleotide variants may be naturally occurring allelic variants, or non-naturally occurring variants. Variant nucleotide sequences preferably exhibit at least about 70%, more preferably at least about 80% and most preferably at least about 90% identity to the recited sequence. Such variant nucleotide sequences will generally hybridize to the recite nucleotide sequence under stringent conditions. As used herein, “stringent conditions” include relatively low salt and/or high temperature conditions, such as provided by 0.02M-0.15M NaCl at temperatures of 50° C. to 70° C. These conditions are particularly selective, and tolerate little, if any, mismatch between the template and target strand.

DNA vaccination involves administering antigen-encoding polynucleotides in vivo or in vitro to induce the production of a correctly folded antigen(s) within the target subject or cells. The introduction of the DNA vaccine will cause to be expressed within those cells the structural protein determinants associated with the antigen and the mutated ADP-ribosyltransferase toxin that is devoid of ADP-ribosyltransferase activity. The processed structural proteins will be displayed on the cellular surface of the transfected cells in conjunction with the Major Histocompatibility Complex (MHC) antigens of the normal cell. Even when cell-mediated immunity is not the primary means of preventing infection, it is likely important for resolving established infections. Furthermore, the structural proteins released by the expressing transfected cells can also be picked up by antigen-presenting cells to trigger systemic humoral antibody responses.

The particular novel DNA vaccines, which co-express an antigen and an adjuvant, employed in the present invention can be engineered preferably using one of the two following configurations.

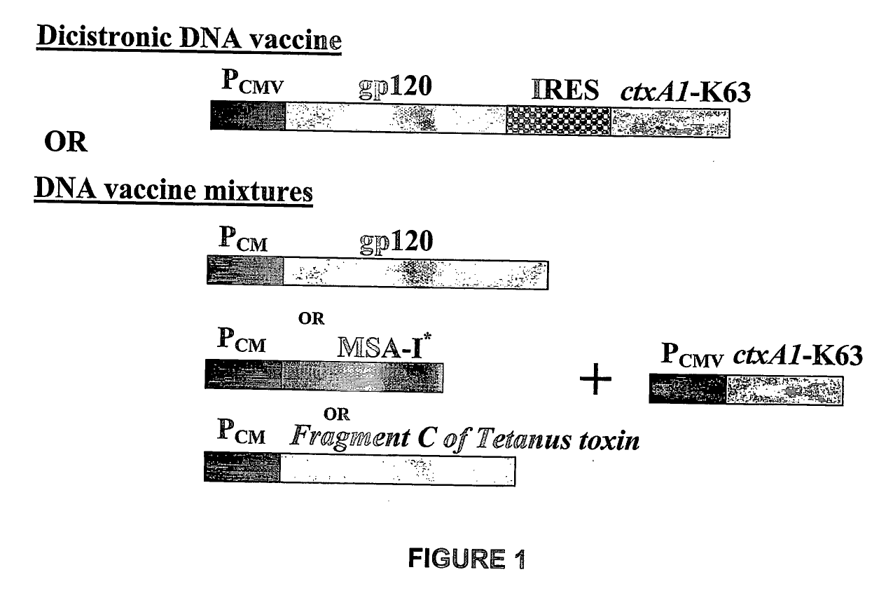

The polynucleotide sequence of DNA vaccines that express an adjuvant comprised of an ADP-ribosyltransferase toxin that is devoid of ADP-ribosyltransferase activity is composed of an expression vector, a eukaryotic promoter, an antigen and an ADP-ribosyltransferase toxin that is devoid of ADP-ribosyltransferase activity such as but not restricted to CT-K63 or LT-K63. Preferably, the nucleotide sequence encoding for the antigen and/or the ADP-ribosyltransferase toxin that is devoid of ADP-ribosyltransferase activity is operatively linked to the promoter. A diagrammatic depiction of generic DNA vaccine configurations that expresses ADP-ribosyltransferase toxin that is devoid of ADP-ribosyltransferase activity is shown in FIG. 1.

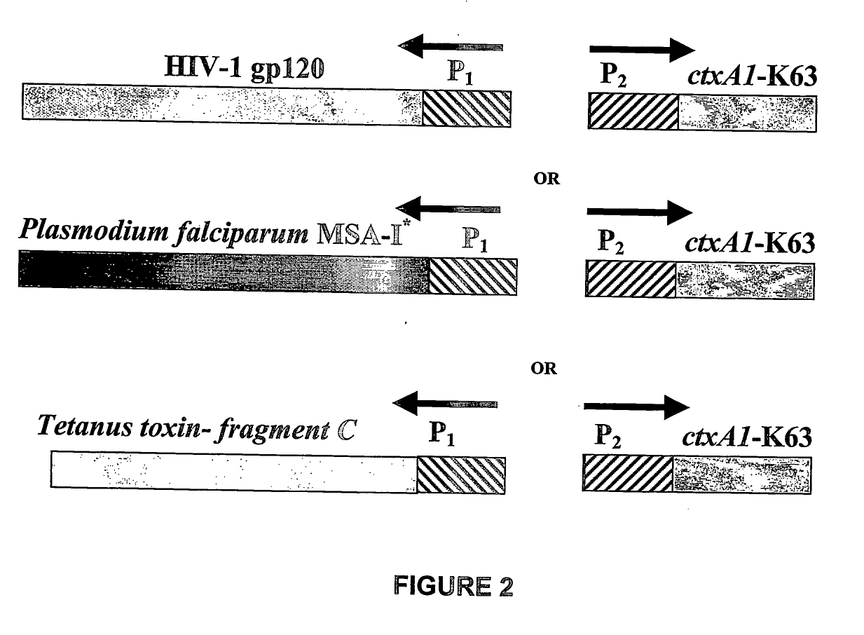

In another configuration, the DNA vaccine that co-expresses an antigen and an ADP-ribosyltransferase toxin that is devoid of ADP-ribosyltransferase activity is composed of an expression vector, two eukaryotic promoters, an ADP-ribosyltransferase toxin that is devoid of ADP-ribosyltransferase activity such as but not restricted to CT-K63 or LT-K63, and at least one vaccine antigen. A diagrammatic depiction of a generic DNA vaccine that expresses ADP-ribosyltransferase toxin that is devoid of ADP-ribosyltransferase activity and an immunogen using two eukaryotic promoters is shown in FIG. 2.

The particular ADP-ribosyltransferase toxin that is devoid of ADP-ribosyltransferase activity may be any derivative of the A subunit of cholera toxin (i.e. CtxA; GenBank accession no. X00171, AF175708, D30053, D30052,), or parts thereof (i.e. the A1 domain of the A subunit of Ctx (i.e. CtxA1; GenBank accession no. K02679)), from any classical Vibrio cholerae (E.g. V. cholerae strain 395, ATCC # 39541) or El Tor V. cholerae (E.g. V. cholerae strain 2125, ATCC # 39050) that lack ADP-ribosyltransferase catalytic activity but retain the structural integrity, including but not restricted to replacement of arginine-7 with lysine (herein referred to as “R7K”), serine-61 with lysine (S61K), serine-63 with lysine (S63K), valine-53 with aspartic acid (V53D), valine-97 with lysine (V97K) or tyrosine-104 with lysine (Y104K), or combinations thereof.

Alternatively, the particular ADP-ribosyltransferase toxin that is devoid of ADP-ribosyltransferase activity may be any derivative of cholera toxin that fully assemble, but are nontoxic proteins due to mutations in the catalytic-site, or adjacent to the catalytic site, respectively. Such mutants are made by conventional site-directed mutagenesis procedures, as described below.

Further, the ADP-ribosyltransferase toxin that is devoid of ADP-ribosyltransferase activity may be any derivative of the A subunit of heat-labile toxin (referred to herein as “LtxA” of enterotoxigenic Eschlerichia coli (GenBank accession # M35581) isolated from any enterotoxigenic Escherichia coli, including but not restricted to E. coli strain H10407 (ATCC # 35401) that lack ADP-ribosyltransferase catalytic activity but retain the structural integrity, including but not restricted to R7K, S61K, S63K, V53D, V97K or Y104K, or combinations thereof. Still further, the particular ADP-ribosyltransferase toxin that is devoid of ADP-ribosyltransferase activity may be any fully assembled derivative of cholera toxin that is nontoxic due to mutations in, or adjacent to, the catalytic site. Such mutants are made by conventional site-directed mutagenesis procedures, as described below.

Mutations that inactivate the catalytic activity of the target ADP-ribosyltransferase toxin (e.g. CtxA and LtxA) can be introduced into gram-negative bacteria using any well-known mutagenesis technique. These include but are not restricted to: (a) non-specific mutagenesis, using chemical agents such as N-methyl-N′-nitro-N-nitrosoguanidine, acridine orange, ethidium bromide, or non-lethal exposure to ultraviolet light (Miller (Ed), 1991, In: A short course in bacterial genetics, Cold Spring Harbor Press, Cold Spring Harbor, N.Y.); (b) Site-Directed mutagenesis by conventional procedures or using QuikChange® Site-Directed Kit (Catalog #200518, Stratagene). The latter site-directed mutagenesis process entails whole-plasmid PCR using the target plasmid (e.g. pOGL1-A1) as template, and forward and reverse primers that modify the target nucleotides (e.g. replace nucleotides 187-189 in CtxA1 (i.e. the serine-63 TCA codon) with a lysine codon (i.e. 5′-AAA); See Examples). The PCR-generated plasmids are digested with DpnI to remove the template DNA and the digested DNA was introduced into E. coli Stable2® by standard transformation procedures. The transformed bacilli are cultured at 30° C. for 16 hr on solid media (e.g. tryptic soy agar; Difco, Detroit Mich.) supplemented with the appropriate antibiotic corresponding to the antibiotic-resistance gene on the target plasmid (e.g. 100-μg/ml ampicillin).

Isolated colonies that grow on the solid media are selected and grown overnight in 3 ml of liquid media (e.g. Luria-Bertani broth, Difco) supplemented with the appropriate antibiotic corresponding to the antibiotic-resistance gene on the target plasmid (e.g. 100-μg/ml ampicillin). Supercoiled plasmid DNA is extracted from the overnight liquid cultures using a Qiagen® Mini Plasmid DNA Preparation Kit (Cat No Q7106). To screen for an appropriate mutant derivative, plasmid preparations are subjected to PCR using primers specific for the mutant ADP-ribosyltransferase allele and the PCR-generated products are analyzed by agarose gel electrophoresis. Clones carrying plasmids that prove positive for mutant ADP-ribosyltransferase allele are stored at −80° C. and used as the source of DNA for the vaccination studies.

Standard procedures are used to construct each mutant ADP-ribosyltransferase allele. Typically, the DNA sequence of each component of a proposed DNA vaccine is downloaded and a plasmid construction strategy is generated using Clone Manager® software version 4.1 (Scientific and Educational Software Inc., Durham N.C.). This software enables the design PCR primers and the selection of restriction endonuclease (RE) sites that are compatible with the specific DNA fragments being manipulated. REs (New England Biolabs Beverly, Mass.), T4 DNA ligase (New England Biolabs, Beverly, Mass.) and Taq polymerase (Life technologies, Gaithersburg, Md.) are used according to the manufacturers' protocols; Plasmid DNA is prepared using small-scale (Qiagen Miniprep® kit, Santa Clarita, Calif.) or large-scale (Qiagen Maxiprep® kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer will be purchased from Lifetechnologies (Gaithersburg, Md.). DNA ligation reactions and agarose gel electrophoresis are conducted according to well-known procedures (Sambrook et al., supra (1989); (Ausubel, et al., supra (1990)).

PCRs are conducted in a Strategene Robocycler, model 400880 (Strategene). Primer annealing, elongation and denaturation times in the PCRs will be set according procedures online in our laboratory (App. 2,3). E. coli strain Sable2® (LifeTechnologies) can serve as the initial host of each new recombinant plasmid. DNA is introduced into E. coli Stable2® by standard transformation procedures (Sambrook et al., supra (1989); (Ausubel, et al., supra (1990)).

Transformed Stable2® bacilli are cultured at 30° C. for 16 hr on solid agar (e.g. tryptic soy agar; Difco, Detroit Mich.) supplemented with the appropriate antibiotic corresponding to the antibiotic-resistance gene on the target plasmid (e.g. 100-μg/ml ampicillin). Isolated colonies that grow on the solid media are selected and grown overnight in 3 to 10 ml of liquid media (e.g. Luria-Bertani broth, Difco) supplemented with the appropriate antibiotic corresponding to the antibiotic-resistance gene on the target plasmid (e.g. 100-μg/ml ampicillin). Supercoiled plasmid DNA is extracted from the overnight liquid cultures using a Qiagen® Mini Plasmid DNA Preparation Kit (Cat No Q7106).

To screen for an appropriate allelic or mutant derivative, plasmid and chromosomal DNA preparations are subjected to PCR using primers specific for the target allele and the PCR-generated products are analyzed by agarose gel electrophoresis. Clones carrying the appropriate alleles and plasmids are stored at −80° C. Dideoxynucleotide sequencing is conducted to verify that the appropriate nucleotides were introduced into the target Salmonella strains, using conventional automated DNA sequencing techniques and an Applied Biosystems automated sequencer, model 373A (Foster City, Calif.).

The expression of immunogens by the modified recombinant DNA vaccines is confirmed by introducing each plasmid into mammalian cells (e.g. Chinese Hamster Ovary cells; ATCC # CCL-61) using standard transfection procedures (Sambrook, et al., supra (1989); (Ausubel, et al., supra (1990)) and a commercially available transfection kit (e.g. the FuGENE® Transfection System; Roche Molecular Biochemicals, Indianapolis, Ind.). Lysates of the transfected cells and culture supernatants are prepared after incubating 72 hr at 37° C. in 5% CO₂, and are fractionated by SDS-polyacrylamide gel electrophoresis and transferred to nitrocellulose filter. Following transfer, the immunogen will be detected on the filter using a standard immunochemical procedure and mAbs specific for the immunogen as primary antibodies, as described by our group previously. Similarly, plasmids that carry wild type, synthetic or mutant ADP-ribosyltransferase alleles (e.g. CtxA1-K63) will be assessed for ADP-ribosylation activity by transiently transfecting mammalian cells (e.g. Chinese Hamster Ovary cells; ATCC # CCL-61) as above and determined the level of cAMP production by transfected cells using a quantitative cAMP colorimetric assay (Amersham, San Francisco, Calif.), as per the manufacture's instructions.

The particular expression vector employed in the present invention may be selected from any of the commercially available expression vectors, such as pcDNA3.1_{ZEO} (Invitrogen Cat.# V790-20), pRc/CMV (Genebank accession E14286) obtained from Invitrogen Corporation (San Diego, Calif.); pNGVL (National Gene Vector Laboratory, University of Michigan, Mich.); pXT1 (Genebank accession M26398)or pSG5 (Genebank accession Af013258), obtained from Stratagene (LaJolla, Calif.); pPUR (Genebank accession U07648) or pMAM (Genebank accession U02443) obtained from ClonTech (Palo Alto, Calif.); pDual (Genbank accession # AF041247); pG51uc (Genbank accession # AF264724); pACT (Genbank accession # AF264723); pBIND (Genbank accession # AF264722); pCI-Neo (Genbank accession # U47120); pCMV-BD (Genbank accession # AF151088); pIRES-P (Genbank accession # Z75185); pRL-CMV (Genbank accession # AF025843), or by adaptation of a publicly or commercially available eukaryotic expression system.

Any promoter which is well-known to be useful for driving expression of genes in animal cells, may be used in the present invention, such as the viral promoters or parts or derivatives thereof, such as the cytomegalovirus immediate early promoter/enhancer (Genebank accession # AF025843) and rous sarcoma virus long terminal repeat (Genebank accession # M83237; Lon et al. Hum. Immunol., 31: 229-235 (1991)) promoters.

Alternatively, the promoter employed in the present invention can be selected from eukaryotic promoters useful for driving expression of genes in animal cells or parts thereof, including but not restricted to the β-casein promoter (Genebank accession # AF194986; Fan et al. Direct submission (2000)), uteroglobin promoter (Genebank accession # NM003357; Hay et al. Am. J. Physiol., 268: 565-575 (1995)), the desmin gene promoter that is only active in muscle cells (Loirat et al., Virology, 260:74 (1999)); the constitutively expressed β-actin promoter (Genebank accession # NM001101; Vandekerckhove and Weber. Proc. Natl. Acad. Sci. U.S.A., 73: 1106-1110 (1978)), ubiquitin (Genebank accession # AJ243268) or the tyrosinase promoter (Genebank accession # NM000372; Shibaharo et al. J. Exp. Med., 156: 403-414 (1988)).

In some situations, the selected promoter is one that is only active in the target cell type. Examples of tissue specific promoters include, but are not limited to, S1- and β-casein promoters which are specific for mammary tissue (Platenburg et al, Trans. Res., 3:99-108 (1994); and Maga et al, Trans. Res., 3:36-42 (1994)); the phosphoenolpyruvate carboxykinase promoter which is active in liver, kidney, adipose, jejunum and mammary tissue (McGrane et al, J. Reprod. Fert., 41:17-23 (1990)); the tyrosinase promoter which is active in lung and spleen cells, but not testes, brain, heart, liver or kidney (Vile et al, Canc. Res., 54:6228-6234 (1994)); the involucerin promoter which is only active in differentiating keratinocytes of the squamous epithelia (Carroll et al, J. Cell Sci., 103:925-930 (1992)); the uteroglobin promoter which is active in lung and endometrium (Helftenbein et al, Annal. N.Y. Acad. Sci., 622:69-79 (1991)); the desmin gene promoter that is only active in muscle cells (Loirat et al., Virology, 260:74 (1999)).

Translation of mRNA in eukaryotic cells requires the presence of a ribosomal recognition signal. Prior to initiation of translation of mRNA in eukaryotic cells, the 5-prime end of the mRNA molecule is “capped” by addition of methylated guanylate to the first mRNA nucleotide residue (Lewin, Genes V, Oxford University Press, Oxford (1994); Darnell et al, Molecular Cell Biology, Scientific American Books, Inc., W.H. Freeman and Co., New York, N.Y. (1990)). It has been proposed that recognition of the translational start site in mRNA by the eukaryotic ribosomes involves recognition of the cap, followed by binding to specific sequences surrounding the initiation codon on the mRNA. After recognition of the mRNA by the ribosome, translation initiates and typically produces a single protein species per mRNA molecule (Lewin, Genes V, Oxford University Press, Oxford (1994); Damell et al, Molecular Cell Biology, Scientific American Books, Inc., W.H. Freeman and Co., New York, N.Y. (1990)).

It is possible for cap independent translation initiation to occur and/or to place multiple eukaryotic coding sequences within a eukaryotic expression cassette if an internal ribosome entry sequence (IRES) is present on the mRNA molecule (Duke et al, J. Virol., 66:1602-1609 (1992)). IRES are used by viruses and occasionally in mammalian cells to produce more than one protein species per mRNA molecule as an alternative strategy to mRNA splicing ((Creancier, et al., J. Cell. Biol., 150:275 (2000); lzquierdo and Cuezva, Biochem. J., 346:849 (2000)).

The particular IRES can be selected from any of the commercially available vectors that contain IRES sequences such as those located on plasmids pCITE4a-c (Novagen, URL:http://www.novagen.com; U.S. Pat. No. 4,937,190); pSLIRES11 (Accession: AF171227; pPV (Accession # Y07702); pSVIRES-N (Accession #: AJ00156); Creancier et al. J. Cell Biol., 10: 275-281 (2000); Ramos and Martinez-Sala, RNA, 10: 1374-1383 (1999); Morgan et al. Nucleic Acids Res., 20: 1293-1299 (1992); Tsukiyama-Kohara et al. J. Virol., 66: 1476-1483 (1992); Jang and Wimmer et al. Genes Dev., 4: 1560-1572 (1990)), or on the Bicistronic retroviral vector (Accession #: D88622); or found in eukaryotic cells such as the Fibroblast growth factor 2 IRES for stringent tissue-specific regulation (Creancier, et al., J. Cell. Biol., 150:275 (2000)) or the Internal-ribosome-entry-site of the 3′-untranslated region of the mRNA for the beta subunit of mitochondrial H⁺-ATP synthase (Izquierdo and Cuezva, Biochem. J., 346:849 (2000)).

The novel DNA vaccines of the present invention encode antigens that may be either foreign antigens or endogenous antigens. The foreign antigen may be a protein, an antigenic fragment or antigenic fragments thereof that originate from viral, bacterial and parasitic pathogens.

Alternatively, the foreign antigen may be encoded by a synthetic gene and may be constructed using conventional recombinant DNA methods (See example 1 for synthetic gene construction procedures); the synthetic gene may express antigens or parts thereof that originate from viral and parasitic pathogens. These pathogens can be infectious in humans, domestic animals or wild animal hosts.

The foreign antigen can be any molecule that is expressed by any viral, bacterial or parasitic pathogen prior to or during entry into, colonization of, or replication in their animal host.

The viral pathogens, from which the viral antigens are derived, include, but are not limited to, Orthomyxoviruses, such as influenza virus (Taxonomy ID: 59771; Retroviruses, such as RSV, HTLV-1 (Taxonomy ID: 39015), and HTLV-II (Taxonomy ID: 11909), Herpesviruses such as EBV Taxonomy ID: 10295); CMV (Taxonomy ID: 10358) or herpes simplex virus (ATCC #: VR-1487); Lentiviruses, such as HIV-1 (Taxonomy ID: 12721) and HIV-2 Taxonomy ID: 11709); Rhabdoviruses, such as rabies; Picornoviruses, such as Poliovirus (Taxonomy ID: 12080); Poxviruses, such as vaccinia (Taxonomy ID: 10245); Rotavirus (Taxonomy ID: 10912); and Parvoviruses, such as adeno-associated virus 1 (Taxonomy ID: 85106).

Examples of viral antigens can be found in the group including but not limited to the human immunodeficiency virus antigens Nef (National Institute of Allergy and Infectious Disease HIV Repository Cat. # 183; Genbank accession # AF238278), Gag, Env (National Institute of Allergy and Infectious Disease HIV Repository Cat. # 2433; Genbank accession # U39362), Tat (National Institute of Allergy and Infectious Disease HIV Repository Cat. # 827; Genbank accession # M13137), mutant derivatives of Tat, such as Tat-Δ31-45 (Agwale et al. Proc. Natl. Acad. Sci. In press. Jul. 8^{th} (2002)), Rev (National Institute of Allergy and Infectious Disease HIV Repository Cat. # 2088; Genbank accession # L14572), and Pol(National Institute of Allergy and Infectious Disease HV Repository Cat. # 238; Genbank accession # AJ237568) and T and Bcell epitopes of gp120 (Hanke and McMichael, AIDS Immunol Lett., 66:177 (1999); Hanke, et al., Vaccine, 17:589 (1999); Palker et al, J. Immunol., 142:3612-3619 (1989)) chimeric derivatives of HIV-1 Env and gp120, such as but not restricted to fusion between gp120 and CD4 (Fouts et al., J. Virol. 2000, 74:11427-11436 (2000)); truncated or modified derivatives of HIV-1 env, such as but not restricted to gp140 (Stamatos et al. J Virol, 79:9656-9667 (1998)) or derivatives of HIV-1 Env and/or gp140 thereof (Binley, et al. J Virol, 76:2606-2616 (2002); Sanders, et al. J Virol, 74-5091-5100 (2000); Binley, et al. J Virol, 74:627-641 (2000)), the hepatitis B surface antigen (Genbank accession # AF043578; Wu et al, Proc. Natl. Acad. Sci., USA, 86:4726-4730 (1989)); rotavirus antigens, such as VP4 (Genbank accession # AJ293721;

Mackow et al, Proc. Natl. Acad. Sci., USA, 87:518-522 (1990)) and VP7 (GenBank accession # AY003871; Green et al, J. Virol., 62:1819-1823 (1988)), influenza virus antigens such as hemagglutinin or (GenBank accession # AJ404627; Pertmer and Robinson, Virology, 257:406 (1999)); nucleoprotein (GenBank accession # AJ289872; Lin et al, Proc. Natl. Acad. Sci., 97: 9654-9658 (2000))) herpes simplex virus antigens such as thymidine kinase (Genbank accession # AB047378; Whitley et al, In: New Generation Vaccines, pages 825-854).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumioniae, Pseudomonas spp., Vibrio spp., and Borellia burgdorferi.

Examples of protective antigens of bacterial pathogens include the somatic antigens of enterotoxigenic E. coli, such as the CFA/I funbrial antigen (Yamamoto et al, Infect. Immun., 50:925-928 (1985)) and the nontoxic B-subunit of the heat-labile toxin (Klipstein et al, Infect. Immun., 40:888-893 (1983)); pertactin of Bordetella pertussis (Roberts et al, Vacc., 10:43-48 (1992)), adenylate cyclase-hemolysin of B. pertussis (Guiso et al, Micro. Path., 11:423-431 (1991)), fragment C of tetanus toxin of Clostridiuni tetani (Fairweather et al, Infect. Immun., 58:1323-1326 (1990)), OspA of Borellia burgdorferi (Sikand, et al. Pediatrics, 108:123-128 (2001); Wallich, et al. Infect Immun, 69:2130-2136 (2001)), protective paracrystalline-surface-layer proteins of Rickettsia prowazekii and Rickettsia typhi (Carl, et al. Proc Natl Acad Sci USA, 87:8237-8241 (1990)), the listeriolysin (also known as “Llo” and “Hly”) and/or the superoxide dismutase (also know as “SOD” and “p60”) of Listeria monocytogenes (Hess, J., et al. Infect. Immun. 65:1286-92 (1997); Hess, J., et al. Proc. Natl. Acad. Sci. 93:1458-1461 (1996); Bouwer, et al. J. Exp. Med. 175:1467-71 (1992)), the urease of Helicobacter pylori (Gomez-Duarte, et al. Vaccine 16, 460-71 (1998); Corthesy-Theulaz, et al. Infection & Immunity 66, 581-6 (1998)), and the receptor-binding domain of lethal toxin and/or the protective antigen of Bacillus anthrax (Price, et al. Infect. Immun. 69, 4094-4515 (2001)).

The parasitic pathogens, from which the parasitic antigens are derived, include but are not limited to, Plasmodium spp., such as Plasmodium falciparum (ATCC#: 30145); Trypanosome spp., such as Trypanosoma cruzi (ATCC#: 50797); Giardia spp., such as Giardia intestinalis (ATCC#: 30888D); Boophilus spp., Babesia spp., such as Babesia microti (ATCC#: 30221); Entamoeba spp., such as Entamoeba histolytica (ATCC#: 30015); Eimeria spp., such as Eimeria maxima (ATCC# 40357); Leishmania spp., (Taxonomy ID: 38568); Schistosome spp., such as Schistosoma mansoni (Genbank accession # AZ301495) Brugia spp., such as Brugia malayi (Genbank accession # BE352806) Fascida spp., such as Fasciola hepatica (Genbank accession # AF286903) Dirofilaria spp., such as Dirofilaria immitis (Genbank accession # AF008300) Wuchereria spp., such as Wuchereria bancrofti Genbank accession # AF250996) and Onchocerea spp; such as Onchocerca volvulus Genbank accession # BE588251).

Examples of parasite antigens can be found in the group including but not limited to the pre-erythrocytic stage antigens of Plasmodium spp. (Sadoff et al, Science, 240:336-337 (1988); Gonzalez, et al., J. Infect. Dis., 169:927 (1994); Sedegah, et al., Proc. Natl. Acad. Sci. 91:9866 (1994); Gramzinski, et al., Vaccine, 15:913 (1997); Hoffman, et al., Vaccine, 15:842 (1997)) such as the circumsporozoite antigen of P. falciparum (GenBank accession # M22982) or P vivax (GenBank accession # M20670); the liver stage antigens of Plasmodium spp. (Hollingdale et al., Ann. Trop. Med. Parasitol., 92:411 (1998), such as the liver stage antigen 1 (as referred to as LSA-1; GenBank accession # AF086802); the merozoite stage antigens of Plasmodium spp. (Holder et al., Parassitologia, 41:409 (1999); Renia et al., Infect. Immun., 65:4419 (1997); Spetzler et al, Int. J. Pept. Prot. Res., 43:351-358 (1994)), such as the merozoite surface antigen-1 (also referred to as MSA-1 or MSP-1; Genank accession # AF199410); the surface antigens of Entamoeba histolytica (Mann et al, Proc. Natl. Acad. Sci., USA, 88:3248-3252 (1991)), such as the galactose specific lectin (GenBank accession # M59850) or the serine rich Entamoeba histolytica protein (also referred to as SREHP; Zhang and Stanley, Vaccine, 18:868 (1999)); the surface proteins of Leishmania spp. (also referred to as gp63; Russell et al, J. Immunol., 140:1274-1278 (1988); Xu and Liew, Immunol., 84: 173-176 (1995)), such as 63 kDa glycoprotein (gp63) of Leishmania major (GenBank accession # Y00647 or the 46 kDa glycoprotein (gp46) of Leishmania major (Handman et al, Vaccine, 18: 3011-3017 (2000); paramyosin of Brugia malayi (GenBank accession # U77590; Li et al, Mol. Biochem. Parasitol., 42:315-323 (1991)), the triose-phosphate isomerase of Schistosoma mansoni (GenBank accession # W0678 1; Shoemaker et al, Proc. Natl. Acad. Sci., USA, 89:1842-1846 (1992)); the secreted globin-like protein of Trichostrongylus colubriformis (GenBank accession # M63263; Frenkel et al, Mol. Biochem. Parasitol, 50:27-36 (1992)); the glutathione-S-transferase's of Fasciola hepatica (GenBank accession # M77682; Hillyer et al, Exp. Parasitol., 75:176-186 (1992)), Schistosoma bovis (Genbank accession # M77682) and S. japonicum (GenBank accession # U58012; Bashir et al, Trop. Geog. Med., 46:255-258 (1994)); Ag 85 A gene of Mycobacterium tuberculosis (GenBank accession # AY207396) or Ag85B (GenBank accession # AY207395); and KLH of Schistosoma bovis and S. japonicum (Bashir et al, supra).

As mentioned earlier, DNA vaccine formulations that direct the coexpression of an antigen and ADP-ribosyltransferase toxin devoid of ADP-ribosyltransferase activity may encode an endogenous antigen, which may be any cellular protein, cytokine, chemokine, or parts thereof, that may be expressed in the recipient cell, including but not limited to tumor antigens, or fragments and/or derivatives of tumor antigens, thereof. Thus, in the present invention, DNA vaccines that co-express an antigen and an adjuvant may encode tumor antigens or parts or derivatives thereof. Alternatively, DNA vaccines that co-express an antigen and an adjuvant may encode synthetic genes, which encode tumor-specific antigens or parts thereof.

Examples of tumor specific antigens include prostate specific antigen (Gattuso et al, Human Pathol., 26:123-126 (1995)), TAG-72 and CEA (Guadagni et al, Int. J. Biol. Markers, 9:53-60 (1994)), human tyrosinase (GenBank accession # M27160; Drexler et al., Cancer Res., 59:4955 (1999); Coulie et al, J. Immunothera., 14:104-109 (1993)), tyrosinase-related protein (also referred to as TRP; GenBank accession # AJ132933; Xiang et al., Proc. Natl. Acad. Sci., 97:5492 (2000)); tumor-specific peptide antigens (Dyall et al., J. Exp. Med., 188:1553 (1998).

The novel DNA vaccines described herein are produced using procedures well known in the art, including polymerase chain reaction (PCR; Sambrook, et al., Molecular cloning; A laboratory Manual: Vol. 1-3, Cold Spring Harbor Laboratory Press, Cold Spring Harbor, N.Y. (1989)); DNA synthesis using an Applied Biosystems DNA synthesizer (Perkin Elmer ABI 3948, using the standard cycle as according to procedures provided by the manufacturer); agarose gel electrophoresis (Ausubel, Brent, Kingston, Moore, Seidman, Smith and Struhl. Current Protocols in Molecular Biology: Vol. 1 and 2, Greene Publishing Associates and Wiley-Interscience, New York (1990)); restriction endonuclease digestion of DNA (Sambrook, et al., supra (1989)); annealing DNA fragments using bacteriophage T4 DNA ligase (New England Biolabs, Cat #202CL; Sambrook, Fritsch, and Maniatis. Molecular cloning; A laboratory Manual: Vol. 1-3, Cold Spring Harbor Laboratory Press, Cold Spring Harbor, N.Y. 1989)); introducing recombinant plasmids into Escherichia coli by electrotransformation (also called electroporation; (Sambrook, et al., supra (1989)); culturing of E. coli isolates that carry recombinant plasmids on solid media (e.g. Tryptic Soy Agar; Beckton Dickenson, Sparks, Md. cat #211046) or in liquid media (e.g. Tryptic Soy Broth; Beckton Dickenson, Sparks, Md. cat #211771) containing the appropriate antibiotics (e.g. 100 μg/ml ampicillin 20 μg/ml chloramphenicol or 50 μg/ml kanamycin) for the selection of bacteria that carry the recombinant plasmid; isolation of plasmid DNA using commercially available DNA purification kits (Qiagen, Santa Clarita, Calif. EndoFree Plasmid Maxi Kit, cat # 12362); transfection of murine and human cells using the FuGENE® proprietary multi-component transfection system using the procedure recommended by the manufacturer (Roche Diagnostics Corporation, Roche Molecular Biochemicals, Indianapolis, Ind. cat # 1 815 091; e.g. Schoonbroodt and Piette, Biochemica 1:25 (1999)); culturing murine or human cells lines in RPMI 1640 medium (Life Technologies, Gaithersburg Md.) containing 10% (v/v) fetal calf serum (Gemini Bioproducts, Calabasas, Calif. cat #100-107; See also Current Protocols in immunology, Greene Publishing Associates and Wiley-Interscience, New York (1990)); analysis of tissue culture supernatants and cell lysates by sodium dodecylsufate-polyacrylamide gel electrophoresis (SDS-PAGE; Harlow and Lane. Using Antibodies, A Laboratory Manual, Cold Spring Harbor Laboratory Press, NY, (1988)) and immunoblotting (Harlow and Lane. Using Antibodies, A Laboratory Manual. Cold Spring Harbor Laboratory Press, NY, (1988)); quantitation of recombinant proteins produced by recombinant plasmids in murine or human cells using a semi-quantitative immunoblot (Abacioglu, Y. H. et al., AIDS Res. Hum. Retroviruses 10:371 (1994)), or a capture enzyme-linked immunosorbent assay (ELISA; Ausubel, et al., Current Protocols in Molecular Biology: Vol. 1 and 2, Greene Publishing Associates and Wiley-Interscience, New York (1990)); quantitative reverse transcriptase (RT)-PCR is conducted as described (Ausubel, et al., In: Current Protocols in Molecular Biology: Vol. 1 and 2, Greene Publishing Associates and Wiley-Interscience, New York (1990)), using the Thermoscript RT-PCR System according to the manufacturer's directions (Life Technologies, Gaithersburg Md.; cat #11146-016).

DNA sequences encoding the individual components of the novel DNA vaccines of the present invention, such as the promoter/enhancer, antigen, internal ribosome entry site (IRESs), and the ADP-ribosyltransferase toxin devoid of ADP-ribosyltransferase activity, may be obtained from the American Type Culture Collection (ATCC, Manassas, Va.). Recombinant bacteria containing the plasmids that encode the genes of interest are cultured as described above; the plasmid DNA is purified and the target sequence is isolated and analyzed by restriction endonuclease digestion or by PCR (Protocols for these procedures are provided above).

Alternatively, in instances where the desired DNA sequence is not available at the ATCC, individual DNA sequences can be made de novo using a DNA sequence obtained from GenBank or from commercial gene databases, e.g. Human Genome Sciences (Gaithersburg, Md.), as the blueprint of the target gene, DNA fragment, or parts thereof. Thus, de novo-generated DNA encoding promoter/enhancers, antigens, internal ribosome entry sites (IRESs), and ADP-ribosyltransferase toxin devoid of ADP-ribosyltransferase activity are synthesized using procedures well known in the art (Andre et al., supra, (1998); et al., Haas supra, (1996)). Briefly, the procedure entails a step-by-step approach, wherein synthetic oligonucleotides 100-200 nucleotides in length (i.e. preferably with sequences at the 5′- and 3′ ends that match at the 5′ and 3′ ends of the oligonucleotides that encodes the adjacent sequence) are produced using an automated DNA synthesizer (E.g. Applied Biosystems ABI™ 3900 High-Throughput DNA Synthesizer (Foster City, Calif. 94404 U.S.A.)). Using the same approach, the complement oligonucleotides are synthesized and annealed with the complementary partners to form double stranded oligonucleotides. Pairs of double stranded oligonucleotides (i.e. those that encode adjacent sequences) and joined by ligation to form a larger fragment. These larger fragments are purified by agarose gel electrophoresis and isolated using a gel purification kit (E.g. The QIAEX® II Gel Extraction System, from Qiagen, Santa Cruz, Calif., Cat. No. 12385). This procedure is repeated until the full-length DNA molecule is created. After each round of ligation the fragments can be amplified by PCR to increase the yield. Procedures for de novo DNA synthesis are well known to the art and are described elsewhere (Andre et al., supra, (1998); et al., Haas supra, (1996)); alternatively synthetic genes can be purchased commercially, e.g. from the Midland Certified Reagent Co. (Midland, Tex.).

Following completion of the de novo gene synthesis the integrity of the coding sequence in the resultant DNA fragment is verified by automated dideoxynucleic acid sequencing using an Applied Biosystems Automated DNA Sequencer or using a commercial facility that has the appropriate capabilities and equipment, such as the Biopolymer Core Facility, University of Maryland, Baltimore Md.

It is understood in the art that certain changes to the nucleotide sequence employed in a genetic construct have little or no bearing on the proteins encoded by the construct, for example due to the degeneracy of the genetic code. Such changes result either from silent point mutations or point mutations that encode different amino acids that do not appreciably alter the behavior of the encoded protein. It is also understood that portions of the coding region can be eliminated without affecting the ability of the construct to achieve the desired effect, namely induction of a protective immune response against poxvirus. It is further understood in the art that certain advantageous steps can be taken to increase the antigenicity of an encoded protein by modifying its amino acid composition. Such changes in amino acid composition can be introduced by modifying the genetic sequence encoding the protein.

The specific method used to purify the DNA vaccines of the present invention is not critical thereto and may be selected from previously described procedures used to purify conventional DNA vaccines (e.g. endotoxin-free large-scale DNA purification kits from Qiagen, Santa Clarita, Calif.; “EndoFree Plasmid Maxi Kit”, cat # 12362), or two rounds of purification using Cesium chloride density gradients (Ausubel, et al., supra (1990)). Endotoxin levels, which are preferably less than 10 Endotoxin Units (i.e. EU) per ml, are measured using one or more of the well-known procedures (E.g. The Limulus Amebocyte Lysate assay (Cape Cod Associates, Cape Cod, Me.; Cat. No. 3P9702); the chicken embryo toxicity assay (Kotani et al., Infect. Immun., 49:225 (1985)); the rabbit pyrogenicity assay (Kotani et al., supra (1985)) and the Schwartzman assay (Kotani et al., supra (1985)).

The specific method used to formulate the novel DNA vaccines described herein is not critical to the present invention and can be selected from previously described procedures used to formulate DNA vaccines, such as formulations that combine DNA vaccine with a physiological buffer (Felgner et al., U.S. Pat. No. 5,589,466 (1996)); aluminum phosphate or aluminum hydroxyphosphate (e.g. Ulmer et al., Vaccine, 18:18 (2000)), monophosphoryl-lipid A (also referred to as MPL or MPLA; Schneerson et al. J. Immunol., 147: 2136-2140 (1991); e.g. Sasaki et al. Inf. Immunol., 65: 3520-3528 (1997); Lodmell et al. Vaccine, 18: 1059-1066 (2000)), QS-21 saponin (e.g. Sasaki, et al., J. Virol., 72:4931 (1998); dexamethasone (e.g. Malone, et al., J. Biol. Chem. 269:29903 (1994); CpG DNA sequences (Davis et al., J. Immunol., 15:870 (1998); lipopolysaccharide (LPS) antagonist (e.g. Hone et al., U.S. Pat. No. 6,368,604 (1997)), an additional plasmid encoding a cytokine (e.g. Hayashi et al. Vaccine, 18: 3097-3105 (2000); Sin et al. J. Immunol., 162: 2912-2921 (1999); Gabaglia et al. J. Immunol., 162: 753-760 (1999); Kim et al., Eur J Immunol., 28:1089 (1998); Kim et al., Eur. J. Immunol., 28:1089 (1998); Barouch et al., J. Immunol., 161:1875 (1998); Okada et al., J. Immunol., 159:3638 (1997); Kim et al., J. Virol., 74:3427 (2000)), and/or an additional plasmid encoding a chemokine (e.g. Boyer et al., Vaccine 17(Suppl 2):S53 (1999); Xin et al., Clin. Immunol., 92:90 (1999)).

The DNA vaccine that direct the coexpression of an antigen and an ADP-ribosyltransferase toxin devoid of ADP-ribosyltransferase activity can be introduced into the animal by intravenous, intramuscular, intradermal, intraperitoneally, intranasal, oral or pulmonary inoculation routes and inoculation by particle bombardment (i.e., gene gun). The specific method used to introduce the DNA vaccines that co-express an antigen and an ADP-ribosyltransferase toxin devoid of ADP-ribosyltransferase activity described herein into the target animal is not critical to the present invention and can be selected from methods well know in the art for such intramuscular, intravenous, intradermal, intraperitoneally, intranasal, oral, pulmonary inoculation routes administration of said vaccines (an extensive database of publications describing the above cited vaccination procedures is located at URL: http://www.DNAvaccine.com/Biblio/articles.html).

Oral inoculation of the target animal with the DNA vaccines that co-expresses and antigen and an adjuvant of the present invention can be achieved using a non-pathogenic or attenuated bacterial DNA vaccine vector (Powell et al., U.S. Pat. No. 5,877,159 (1999); Powell et al., U.S. Pat. No. 6,150,170). The amount of the bacterial DNA vaccine vector of the present invention to be administered will vary depending on the species of the subject, as well as the disease or condition that is being treated. Generally, the dosage employed will be about 10³ to 10¹¹ viable organisms, preferably about 10³ to 10⁹ viable organisms, as described (Shata et al., Vaccine 20:623-629 (2001); Shata and Hone, J. Virol. 75:9665-9670 (2001)).

The bacterial DNA vaccine vector carrying the DNA vaccine of the present invention is generally administered along with a pharmaceutically acceptable carrier or diluent. The particular pharmaceutically acceptable carrier or diluent employed is not critical to the present invention. Examples of diluents include a phosphate buffered saline, buffer for buffering against gastric acid in the stomach, such as citrate buffer (pH 7.0) containing sucrose, bicarbonate buffer (pH7.0) alone (Levine et al, J. Clin. Invest., 79:888-902 (1987); and Black et al J. Infect. Dis., 155:1260-1265 (1987)), or bicarbonate buffer (pH 7.0) containing ascorbic acid, lactose, and optionally aspartame (Levine et al, Lancet, II:467-470 (1988)). Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10% (w/v).

To deliver DNA vaccines by particle bombardment, the PowderJect-XR.TM. gene gun device described in WO 95/19799, Jul. 27, 1995 may be used. Other instruments are available and known to people in the art. This instrument, delivers DNA-coated gold beads directly into epidermal cells by high-velocity particle bombardment.

The technique of accelerated particles gene delivery or particle bombardment is based on the coating of DNA to be delivered into cells onto extremely small carrier particles, which are designed to be small in relation to the cells sought to be transformed by the process. The DNA sequence containing the desired genes can be simply dried onto a small inert particle. The particle may be made of any inert material such as an inert metal (gold, silver, platinum, tungsten, etc.) or inert plastic (polystyrene, polypropylene, polycarbonate, etc.). Preferably, the particle is made of gold, platinum or tungsten. Most preferably, the particle is made of gold. Suitably, the particle is spherical and has a diameter of 0.5 to 5 microns, preferably 1 to 3 microns. DNA molecules in such a form may have a relatively short period of stability and may tend to degrade rather rapidly due to chemical reactions with the metallic or oxide substrate of the particle itself. Thus, if the carrier particles are first coated with an encapsulating agent, the DNA strands have greatly improved stability and do not degrade significantly even over a time period of several weeks. A suitable encapsulating agent is polylysine (molecular weight 200,000) which can be applied to the carrier particles before the DNA molecules are applied. Other encapsulating agents, polymeric or otherwise, may also be useful as similar encapsulating agents, including spermidine. The polylysine is applied to the particles by rinsing the gold particles in a solution of 0.02% polylysine and then air-drying or heat drying the particles thus coated. Once the metallic particles coated with polylysine were properly dried, DNA strands are then loaded onto the particles.

The DNA is loaded onto the particles at a rate of between 0.5 and 30 micrograms of DNA per milligram of gold bead spheres. A preferable ratio of DNA to gold is 0.5-5.0 ug of DNA per milligram of gold.

A sample procedure begins with gamma irradiated (preferably about 30 kGy) tefzel tubing. The gold is weighed out into a microfuge tube, spermidine (free base) at about 0.05 M is added and mixed, and then the DNA is added. A 10% CaCl solution is incubated along with the DNA for about 10 minutes to provide a fine calcium precipitate. The precipitate carries the DNA with it onto the beads. The tubes are microfuged and the pellet resuspended and washed in 100% ethanol and the final product resuspended in 100% ethanol at 0.0025 mg/ml PVP. The gold with the DNA is then applied onto the tubing and dried.

The general approach of accelerated particle gene transfection technology is described in U.S. Pat. No. 4,945,050 to Sanford. An instrument based on an improved variant of that approach is available commercially from PowderJect Vaccines, Inc., Madison Wis., and is described in WO 95/19799. Briefly, the DNA-coated particles are deposited onto the interior surface of plastic tubing which is cut to a suitable length to form sample cartridges. A sample cartridge is placed in the path of a compressed gas (e.g., helium at a pressure sufficient to dislodge the particles from the cartridge e.g., 350-400 psi). The particles are entrained in the gas stream and are delivered with sufficient force toward the target tissue to enter the cells of the tissue. Further details are available in the published apparatus application.

The coated carrier particles are physically accelerated toward the cells to be transformed such that the carrier particles lodge in the interior of the target cells. This technique can be used either with cells in vitro or in vivo. At some frequency, the DNA which has been previously coated onto the carrier particles is expressed in the target cells. This gene expression technique has been demonstrated to work in prokaryotes and eukaryotes, from bacteria and yeasts to higher plants and animals.

Thus, the accelerated particle method provides a convenient methodology for delivering genes into the cells of a wide variety of tissue types, and offers the capability of delivering those genes to cells in situ and in vivo without any adverse impact or effect on the treated individual. Therefore, the accelerated particle method is also preferred in that it allows a DNA vaccine capable of eliciting an immune response to be directed both to a particular tissue, and to a particular cell layer in a tissue, by varying the delivery site and the force with which the particles are accelerated, respectively. This technique is thus particularly suited for delivery of genes for antigenic proteins into the epidermis.

A DNA vaccine can be delivered in a non-invasive manner to a variety of susceptible tissue types in order to achieve the desired antigenic response in the individual. Most advantageously, the genetic vaccine can be introduced into the epidermis. Such delivery, will produce a systemic humoral immune response.

To obtain additional effectiveness from this technique, it may also be desirable that the genes be delivered to a mucosal tissue surface, in order to ensure that mucosal, humoral and cellular immune responses are produced in the vaccinated individual. There are a variety of suitable delivery sites available including any number of sites on the epidermis, peripheral blood cells, i.e. lymphocytes, which could be treated in vitro and placed back into the individual, and a variety of oral, upper respiratory, and genital mucosal surfaces.

Gene gun-based DNA immunization achieves direct, intracellular delivery of DNA, elicits higher levels of protective immunity, and requires approximately three orders of magnitude less DNA than methods employing standard inoculation. Moreover, gene gun delivery allows for precise control over the level and form of antigen production in a given epidermal site because intracellular DNA delivery can be controlled by systematically varying the number of particles delivered and the amount of DNA per particle. This precise control over the level and form of antigen production may allow for control over the nature of the resultant immune response.

The methods of the present invention are considered effective if DNA vaccination reduces the severity of the disease symptoms. It is preferred that the immunization method be at least 20% effective in preventing death in an immunized population after challenge with antigen. More preferably, the vaccination method is 50% or more effective, and most preferably 70-100% effective, in preventing death in an immunized population.

Generally, the DNA vaccine administered may be in an amount of about 0.01-10 ug of DNA per dose and will depend on the subject to be treated, capacity of the subject's immune system to develop the desired immune response, and the degree of protection desired. Precise amounts of the vaccine to be administered may depend on the judgment of the practitioner and may be peculiar to each subject and antigen.

The vaccine for eliciting an immune response may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. Examples of suitable immunization schedules include: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

In another embodiment, the present invention provides reagents useful for carrying out the present process. Such reagents comprise a DNA fragment containing at least one antigen and an ADP-ribosyltransferase toxin that is devoid of ADP-ribosyltransferase activity. Preferably, the DNA is frozen or lyophilized, and the small, inert, dense particle is in dry powder. If a coating solution is used, the dry ingredients for the coating solution may be premixed and premeasured and contained in a container such as a vial or sealed envelope.

The present invention also provides kits that are useful for carrying out the present invention. The present kits comprise a first container means containing the above-described frozen or lyophilized DNA. The kit also comprises a second container, which contains the coating solution or the premixed, premeasured dry components of the coating solution. The kit also comprises a third container means which contains the small, inert, dense particles in dry powder form or suspended in 100% ethanol. These container means can be made of glass, plastic or foil and can be a vial, bottle, pouch, tube, bag, etc. The kit may also contain written information, such as procedures for carrying out the present invention or analytical information, such as the amount of reagent (e.g. moles or mass of DNA) contained in the first container. The written information may be on any of the first, second, and/or third container means, and/or a separate sheet included, along with the first, second, and third container means, in a fourth container. The fourth container means may be, e.g. a box or a bag, and may contain the first, second, and third container.

The following examples are provided for illustrative purposes only, and are in no way intended to limit the scope of the present invention.

### EXAMPLE 1

Recombinant DNA Procedures

Reagents, Bacterial Strains and Plasmids

Restriction endonucleases (New England Biolabs Beverly, Mass.), T4 DNA ligase (New England Biolabs, Beverly, Mass.) and Taq polymerase (Life technologies, Gaithersburg, Md.) were used according to the manufacturers' protocols; Plasmid DNA was prepared using small-scale (Qiagen Miniprep® kit, Santa Clarita, Calif.) or large-scale (Qiagen Maxiprep® kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer were purchased from Life technologies, Gaithersburg, Md. DNA ligation reactions and agarose gel electrophoresis were conducted according to well-known procedures (Sambrook, et al., supra (1989); (Ausubel, et al., supra (1990)).

PCR primers were purchased from the University of Maryland Biopolymer Facility (Baltimore, Md.) and were synthesized using an Applied Biosystems DNA synthesizer (model 373A). PCR primers were used at a concentration of 200 μM and annealing temperatures for the PCR reactions were determined using Clone manager software version 4.1 (Scientific and Educational Software Inc., Durham N.C.). PCRs were conducted in a Strategene Robocycler, model 400880 (Strategene, La Jolla, Calif.). Annealing, elongation and denaturation times in the PCRs were set according to well-known procedures.

Nucleotide sequencing to verify the DNA sequence of each recombinant plasmid described in the following examples was accomplished by conventional automated DNA sequencing techniques using an Applied Biosystems automated sequencer, model 373A.

Escherichia coli strain Sable2® was purchased from Life Technologies (Bethesda, Md.) and served as host of the recombinant plasmids described in the examples below.

Plasmid pCVD002 (Lochman and Kaper, J. Biol. Chem., 258:13722 (1983)) served as a source of the CtxA1-encoding sequences (kindly provided by Dr. Jim Kaper, Department of Microbiology and Immunology, University of Maryland, Baltimore).

Recombinant plasmids were introduced into E. coli strain Stable2® by electroporation using a Gene Pulser (BioRad Laboratories, Hercules, Calif.) set at 200 Ω, 25 μF and 2.5 kV as described (Hone, et al., Vaccine, 9:810 (1991)).

Bacterial strains were grown on tryptic soy agar (Difco, Detroit Mich.) or in tryptic soy broth

(Difco, Detroit Mich.), which were made according to the manufacturer's directions. Unless stated otherwise, all bacteria were grown at 37° C. When appropriate, the media were supplemented with 100 μg/ml ampicillin (Sigma, St. Louis, Mo.).

Bacterial strains were stored at −80° C. suspended in tryptic soy broth containing 30% (v/v) glycerol at ca. 10⁹ colony-forming units (herein referred to as “cfu”) per ml. Plasmid pCITE4a, which contains the IRES of equine encephalitis virus, was purchased from Novagen (Madison Wis.). Plasmid pcDNA3.1_{ZEO}, which contains the colE1 replicon, an ampicillin-resistance allele, the CMV immediate-early promoter, a multicloning site and the bovine hemoglobin poly-adenosine sequence, was purchased from Clonetech (Palo Alto, Calif.). Plasmid pEF1a-syngp120MN carrying synthetic DNA encoding HIV-1_{MN} gp120 (referred to herein as hgp120), in which the native HIV-1 leader peptide was replaced by the human CD5 leader peptide and the codons are optimized for expression in mammalian cells is described elsewhere (Andre et al., supra, (1998); et al., Haas supra, (1996)).

Restriction endonuclease digestion, ligation, and plasmid DNA preparation techniques were all conducted as described earlier. Nucleotide sequencing to verify the structure of each recombinant plasmid described in the following examples was accomplished by standard automated sequencing techniques (Applied Biosystems automated sequencer, model 373A).

### EXAMPLE 2

Vaccination and Immunological Procedures

Source of laboratory animals and handling: BALB/c and C57B1/6 mice aged 6-8 weeks were obtained from Charles River (Bar Harbor, Me.). All of the mice were certified specific-pathogen free and upon arrival at the University of Maryland Biotechnology Institute Animal Facility were maintained in a microisolator environment and allowed to fee and drink ad lib.

Vaccination procedures: Groups of 6 mice were vaccinated intramuscularly with 1-100 μg of endotoxin-free (<0.5 EU per mg of DNA) plasmid DNA suspended in saline (0.85% (w/v) NaCl), as described (Felgner et al., U.S. Pat. No. 5,589,466 (1996)). Booster vaccinations were given using the same formulation, route and dose as used to prime the mice; the spacing of the doses is outlined below.

Serum enzyme-linked immunosorbent assays (ELISAs): Blood (ca. 100 μl per mouse) was collected before and at weekly intervals after vaccination. The presence of gp120-specific IgG in pooled sera collected from the vaccinated mice was determined by ELISA. Aliquots (0.3 μg suspended in 100 μl PBS, pH 7.3) of purified glycosylated HIV-1_{MN} gp120 (Virostat, Portland) were added to individual wells of 96-well Immulon plates (Dynex technologies Inc, Virginia, USA). After incubating 16-20 hr at 4° C., the plates were washed three times with washing buffer (Kirkegaard and Perry Laboratories, Gaithersburg, Md.) and 200 μl of blocking buffer (Kirkegaard and Perry Laboratories, Gaithersburg, Md., USA) was added and the plates were incubated for 1 hr at 25° C. After the blocking was complete, duplicated sets of each serum sample were diluted serially in 3-fold increments (Starting at 1:10) in blocking buffer and incubated for 1 hr at room temperature. Then, the plates were washed six times with washing buffer and 100 μl of horseradish peroxidase-labelled goat anti-mouse IgG (Sigma Immunochemicals, USA), diluted in 1/2000 in blocking buffer, was added to each well and the plates were incubated for 1 hr at 25° C. The plates were washed an additional six times with washing buffer and 100 μl of ABTS substrate (Kirkegaard and Perry Laboratories, Gaithersburg, Md., USA) was added and the plates were incubated for 30 min at 25° C. The absorbance was measured at 405 nm using a Wallac Dynamic Reader, model 1420 (Turku, Finland). A similar procedure was conducted to measure gp120-specific IgG subtypes, IgG1, IgG2a and IgG2b, except that rat anti-mouse IgG1, IgG2a, and IgG2b antibodies conjugated to horseradish peroxidase (diluted 1:8000, 1:2000 and 1:1000, respectively; BioSource International, Keystone, USA) were using in place of the goat anti-mouse IgG.

### EXAMPLE 3

Construction of DNA vaccines encoding an antigen and ADP-ribosyltransferase toxin devoid of ADP-ribosyltransferase activity:

In this example a novel DNA vaccine was constructed, herein designated pOGL1-A1-K63, which co-expresses an antigen (i.e. gp120 of HIV-_{MN}) and a mutant derivative of the A1 domain of the A subunit of Ctx (referred to herein as “CtxA1”) that harbors a lysine substitution at amino acid no. 63 (i.e. herein referred to as “CtxA1-K63”) in place of the serine that is present in the parental CtxA1. Expression vector pcDNA3.1_{ZEO} was purchased from Invitrogen (Carlsbad, Calif.) and carries the CMV promoter that is active in a wide spectrum of eukaryotic cells.

Construction of DNA vaccine pOGL1 was achieved by PCR-amplifying hgp120 from a plasmid pEF1α-syngp120_{MN} (Andre et al., supra, (1998); et al., Haas supra, (1996)) using forward primer 5′-GGGGGGGGATCCATGCCCATGGGGTCTCTGCAACCGCTG (SEQ ID NO. 1) and reverse primer 5′-GGGGGCGGCCGCTTATTAGGCGCGCTTCTCGCGCTGCACCACGCG (SEQ ID NO. 2) using the PCR procedure outlined in example 1 above. The resultant PCR-generated DNA fragment was digested with restriction endonucleases BamHI and NotI and annealed (E.g. by ligation with T4 ligase) with BamHI- and NotI-digested pcDNA3.1_{ZEO} DNA (Invitrogen, Carlsbad, Calif., Cat. No. V860-20). The ligated DNA was introduced into E. coli strain Stable2® (Life Technologies, Gaithersburg, Md.) by electroporation. Plasmid DNA was prepared from 2 ml liquid cultures of individual clones and used to screen for a clone that carried a plasmid with the appropriate restriction endonuclease digestion pattern. One such clone, referred to herein as “H1058”, containing the desired plasmid (referred to herein as “pOGL1”), which is pcDNA3.1_{ZEO} containing the BamH-NotI hgp120 fragment, was stored at −80° C. Additional analysis by restriction endonuclease digestion, PCR of the hgp120 DNA, and dideoxynucleotide sequencing of the cloned hgp120 DNA in pOGL1 was conducted to verify that the hgp120 DNA was not altered during construction.

DNA encoding the IRES of equine encephalitis virus, herein referred to as the cap-independent translational enhancer (U.S. Pat. No. 4,937,190), was amplified from plasmid pCITE4a (Novagen, Madison Wis.; Cat. No. 69912-1; U.S. Pat. No. 4,937,190) using forward primer 5′-ATAAGAATGCGGCCGCTAAGTAAGTAACTTAAGTTCCGGTTATTTTCCACGATA TTGCCGTCTTTTGGCAA (SEQ ID NO. 3) and reverse primer 5′-GCCAAATACATGGCCATATTATCATCGTGTTTTTCAAAGGAA (SEQ ID NO. 4). DNA encoding CtxA1-K63 was amplified from plasmid pOGL1-A1 [13], which has a copy of CtxA1. The nucleotide sequence of ctxA1-K63 was obtained from GenBank (Accession # A16422) and modified by replacing the serine-63 TCA codon (nucleotides 187-189; See sequence above) with a lysine codon (i.e. AAA). The mutant derivative of CtxA1, CtxA1-K63,

```
Nucleotide sequence of CtxA1-K63
(SEQ ID NO. 5)
    1 AATGATGATA AGTTATATCG GGCAGATTCT AGACCTCCTG ATGAAATAAA GCAGTCAGGT
  61 GGTCTTATGC CAAGAGGACA GAGTGAGTAC TTTGACCGAG GTACTCAAAT GAATATCAAC
121 CTTTATGATC ATGCAAGAGG AACTCAGACG GGATTTGTTA GGCACGATGA TGGATATGTT
181 TCCACCAAAA TTAGTTTGAG AAGTGCCCAC TTAGTGGGTC AAACTATATT GTCTGGTCAT
241 TCTACTTATT ATATATATGT TATAGCCACT GCACCCAACA TGTTTAACGT TAATGATGTA
301 TTAGGGGCAT ACAGTCCTCA TCCAGATGAA CAAGAAGTTT CTGCTTTAGG TGGGATTCCA
361 TACTCCCAAA TATATGGATG GTATCGAGTT CATTTTGGGG TGCTTGATGA ACAATTACAT
421 CGTAATAGGG GCTACAGAGA TAGATATTAC AGTAACTTAG ATATTGCTCC AGCAGCAGAT
481 GGTTATGGAT TGGCAGGTTT CCCTCCGGAG CATAGAGCTT GGAGGGAAGA GCCGTGGATT
541 CATCATGCAC CGCCGGGTTG TGGGAATGCT CCAAGATCAT CG_{END}
```

was generated using the QuikChange® Site-Directed Mutagenesis Kit (Catalog #200518, Stratagene). The site-directed mutagenesis process entailed whole-plasmid PCR using pOGL1-A1 DNA as template, forward primer 5′-TGTTTCCCACCAAAATTAGTTTGAGAAGTGC (SEQ ID NO. 6) and reverse primer 5′-CAAACTAATTTTGGTGGAAACATATCCATC (SEQ ID NO. 7); this procedure modified nucleotides 187-189 by replacing TCA (i.e. serine-63 codon) with a lysine codon (i.e. 5′-AAA). The resultant PCR-generated plasmid was digested with DpnI to remove the template DNA and the digested DNA was introduced into E. coli Stable2® by chemical transformation. The transformed bacilli were cultured on tryptic soy agar (Difco, Detroit Mich.) supplemented with 100-μg/ml ampicillin at 30° C. for 16 hr.

Isolated colonies were selected and grown overnight in 3 ml of LB medium supplemented with 100 μg/ml ampicillin. DNA was extracted from overnight liquid cultures using a Qiagen mini plasmid DNA preparation kit (Cat No Q7106). Plasmid PCR using primers specific for CtxA1-K63, and agarose gel electrophoresis were conducted to screen for an appropriate derivative. Several clones tested positive for CtxA1-K63 insert and strain containing the appropriate plasmid (herein referred to as “pOGL1-A1-K63”) were stored at −80° C. as described above. One such isolate was used as the source of pOGL1-A1-K63 DNA for the vaccination studies below.

### EXAMPLE 4

Immunogenicity of a DNA vaccine that directs the coincident expression of an gp120 and ADP-ribosyltransferase toxin devoid of ADP-ribosyltransferase activity

The adjuvant activity of CtXA1-K63 in DNA vaccine pOGL1-A1-K63 was characterized by comparing the immunogenicity of DNA vaccine pOGL1 that expresses gp120 alone, to that of bicistronic DNA vaccine pOGL1-A1-K63 that expresses both gp120 and CtxA1-K63 in BALB/c mice. Accordingly, groups of 3 BALB/c mice were vaccinated intramuscularly with three 40 μg-doses of endotoxin-free plasmid DNA on days 0, 14 and 42. A negative control group of 3 BALB/c mice received three dose 40 μg-doses of plasmid pcDNA3.1 DNA using the same protocol and intervals between doses.

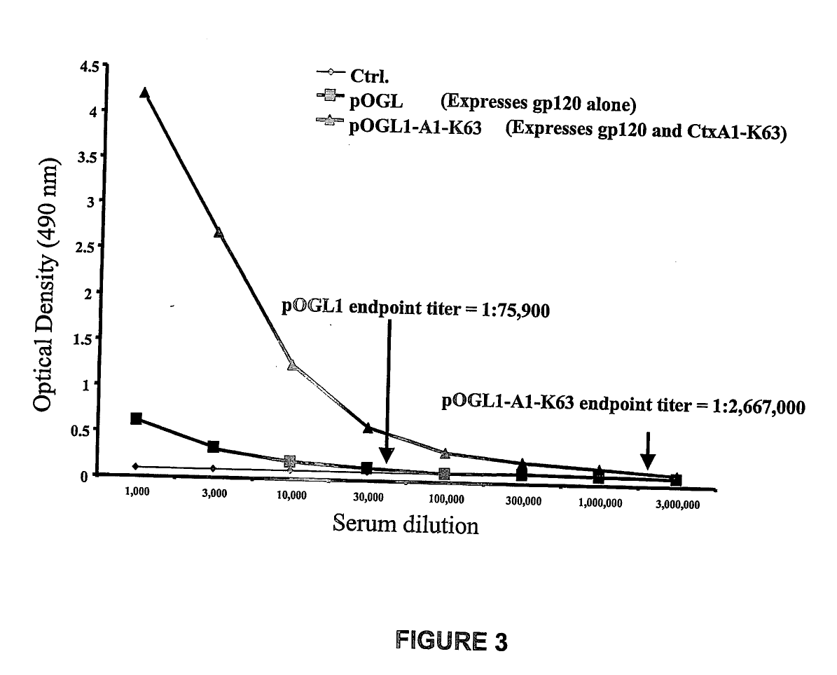

Sera were collected before and at regular intervals after vaccination, and used to measure the serum IgG response against HIV-1_{MN} gp120 by ELISA (Example 2). This experiment demonstrates that mice vaccinated with bicistronic DNA vaccine pOGL1-A1-K63 developed a serum IgG response against gp120 that was significantly greater and remained elevated longer than the analogous serum IgG response in mice vaccinated with the DNA vaccine that expressed gp120 alone (i.e. pOGL1; FIG. 3).

### EXAMPLE 5

Interpretation of the Results

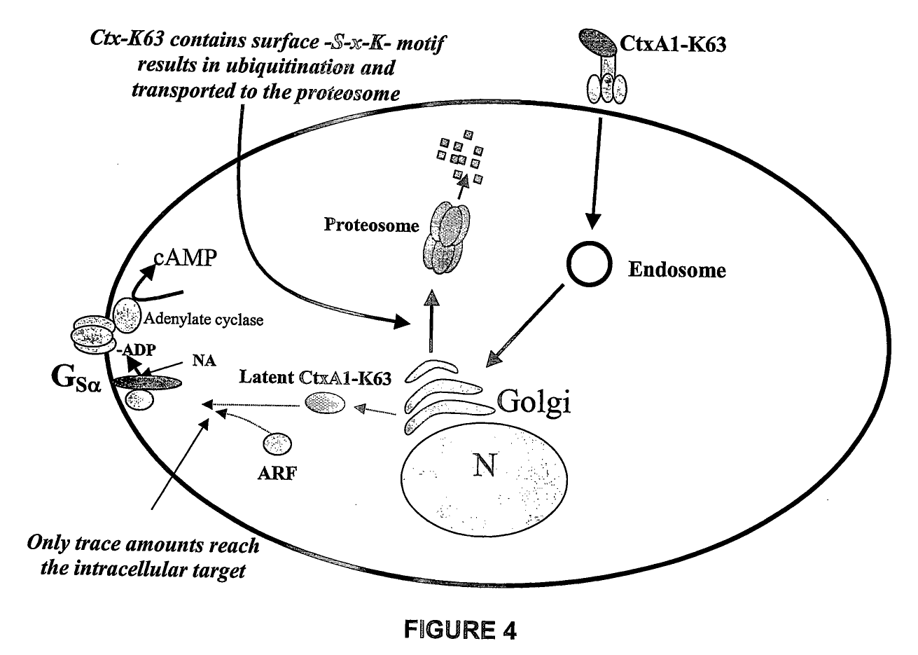

The above unanticipated finding indicates that in contrast to the mutant CT-K63 holotoxin (i.e the protein form) which displays little adjuvant activity, DNA vaccines that express CtxA1-K63 retain potent adjuvanticity. Although not wanting to be held to this theory, it is believed that the basis for this difference is likely the result of differences in the intracellular trafficking pathways employed by the purified holotoxin, compared to CtxA1-K63 when expressed by a DNA vaccine. The mutant CT-K63 holotoxin when added in the form of a purified protein must traffic via the golgi apparatus to reach the cell cytoplasm and during this transport is exposed to the cellular ubiquitination/proteosome degradation machinery (FIG. 4). The presence of the surface-exposed lysine (i.e. K63) serves as a cognate recognition motif for ubiquitination and proteosome The mutant CT-K63 holotoxin when added in the form of a purified protein must traffic via the golgi apparatus to reach the cell cytoplasm and during this transport is exposed to the cellular ubiquitination/proteosome degradation machinery (FIG. 4). The presence of the surface-exposed lysine (i.e. K63) serves as a cognate recognition motif for ubiquitination and proteosome degradation, substantially preventing interaction between the A1-K63 subunit of the mutant holotoxin with the host ADP-ribosyltransferase factor (herein referred to as ARF), and the subsequent ADP-ribosylation of G_{Sα} and adenylate cyclase degradation, substantially preventing interaction between the A1-K63 subunit of the mutant holotoxin with the host ADP-ribosyltransferase factor (herein referred to as ARF), and the subsequent ADP-ribosylation of G_{Sα} and adenylate cyclase (FIG. 4). The reduced ability to reach the host ARF and activate G_{sα} explains why mutants of cholera toxin mutants that carry amino acid substitutions that are recognized by the host ubiquitination and proteosome degradation apparatus (e.g., K63) display relatively insipient adjuvant activity, relative to wild-type cholera toxin [14].

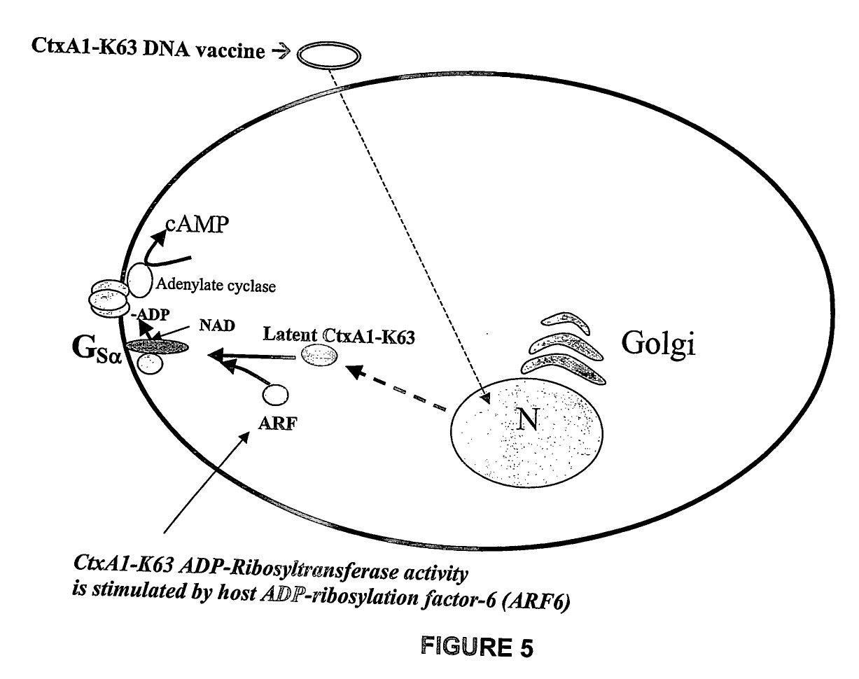

In contrast, expression of CtxA1-K63 by a DNA vaccine bypasses the golgi apparatus, thereby avoiding ubiquitination and proteosome degradation, and allowing access to the endogenous ARFs and G_{sα} (FIG. 5). Be that as it may, said mutants such as CtxA1-K63, are incapable of binding NAD and thus the capacity to access endogenous ARFs and G_{sα} should be insufficient to restore ADP-ribosyltransferase activity. It is known that CtxA1-K63 and related cholera toxin mutants retain the ability to interact with endogenous ARFs (Stevens, et al., Infect. Immun. 67:259-265 (1999)).

The role this interaction in the adjuvant properties of ADP-ribosyltransferase toxins, however, has not heretofore been evaluated. The interaction between CtxA1 and ARF augments the ADP-ribosyltransferase catalytic activity and substantially increases cyclic-adenosine monophosphate (herein referred to as “cAM”) production in treated cells; thus this interaction is required for maximal ADP-ribosyltransferase activity of wild type CT (Jobling, et al., Proc Natl Acad Sci USA, 97:14662-14667 (2000)). It is important to note that the ability to increase cAMP levels is central to the capacity of ADP-ribosyltransferase toxins to impart toxicity [14]. However, the role of the interaction between ADP-ribosyltransferase toxins and ARF in the adjuvanticity of these molecules heretofore remained unknown, since purified mutant ADP-ribosyltransferase toxins that are incapable of binding NAD, such as cholera toxin-K63, were also found to be devoid of ADP-ribosyltransferase activity in host cells and displayed poor adjuvant properties [14,15].

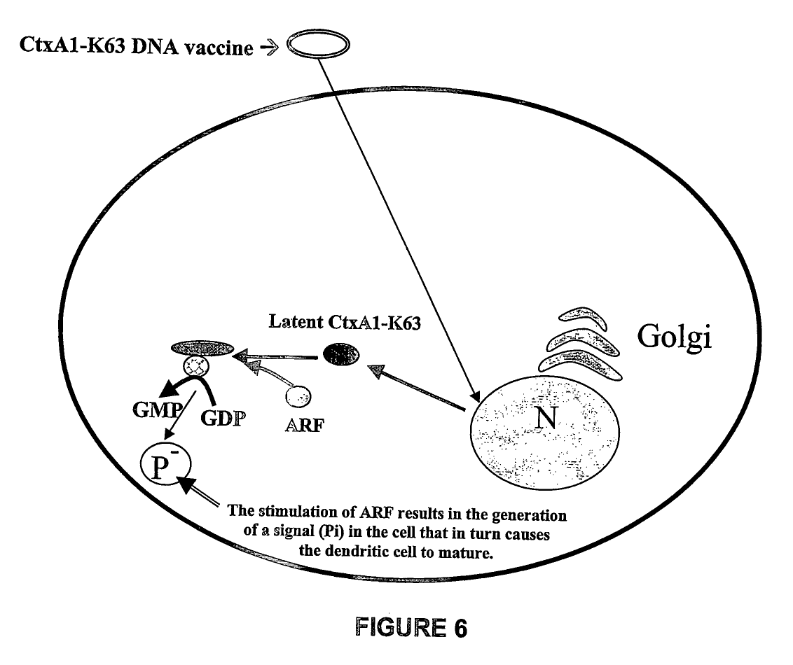

On the other hand, example 4 presents a novel and unexpected finding that delivery of a mutant ADP-ribosyltransferase toxin and that is incapable of binding NAD to the appropriate cellular compartment displays in significant adjuvant activity. Presumably expression CtxA1-K63 by a DNA vaccine in dendritic cells (i.e. Dendritic cells are the key antigen presenting cell involved in promoting DNA vaccine-induced immune responses [16-18]) causes said cells to differentiate into a mature antigen presenting cells, thereby augmenting the immunogenicity of an immunogen that is coincidently expressed with said mutant (Stevens, et al., Infect Immun 67:259-265 (1999); Randazzo, et al., J Biol Chem 268:9555-63 (1993); Jobling and Holmes, Proc. Natl. Acad. Sci. 97:14662-14667 (2000); Zhu, et al., Biochemistry 40:4560-4568 (2001)). One mechanism through which CtxA1-K63 DNA vaccine retains adjuvant activity may be a conformational change following the interaction between CtxA1-K63 and the host ARF, thereby opening the NAD-binding cleft in said mutant toxin (FIG. 5). A more likely scenario is that the binding of CtxA1 to ARF may stimulate the GTPase activity of ARF; the activated ARF may then produce a signal that results in differentiation of dendritic cells that harbor the DNA vaccine into a mature antigen presenting cell, which in turn promote the profound humoral responses to the DNA vaccine-encoded immunogen (FIG. 6).

A key advantage possessed by DNA vaccines that express an ADP-ribosyltransferase toxin devoid of intrinsic ADP-ribosyltransferase activity is that such vaccines are likely to have a broader safety profile in large population studies. In addition, the growth of strains harboring DNA vaccines that express an ADP-ribosyltransferase toxin devoid of intrinsic ADP-ribosyltransferase activity have proven to be more stable and capable of growing the greater optical densities. Thus, strains harboring said mutant DNA vaccine produce about 4-fold more viable bacilli per ml of culture (i.e. for 16 hr at 37° C. in LB with agitation), compared to parallel cultures of strains that carrying a DNA vaccine that expresses a wild type ADP-ribosyltransferase toxin. This finding has obvious manufacturing implications and bodes well for the application of this technology to large-scale public health vaccination programs.

While the invention has been described in detail, and with reference to specific embodiments thereof, it will be apparent to one of ordinary skill in the art that various changes and modifications can be made therein without departing from the spirit and scope thereof.

### References:

The disclosures of the following references and all references cited herein are hereby incorporated herein by reference for all purposes.
1. Hone, D., Morona, R., Attridge, S. & Hackett, J. Construction of defined galE mutants of Salmonella for use as vaccines. J. Infect. Dis. 1987, 156(1), 167-174.
2. Hone, D., Attridge, S., van den Bosch, L. & Hackett, J. A chromosomal integration system for stabilization of heterologous genes in Salmonella-based vaccine strains. Microbial. Path. 1988, 5(6), 407-418.
3. Hone, D. M., Harris, A. M., Chatfield, S., Dougan, G. & Levine, M. M. Construction of genetically defined double aro mutants of Salmonella typhi. Vaccine 1991, 9(11), 810-816.
4. Hone, D. M., Harris, A. M., Lini, V. & Levine, M. M. Construction and characterization of isogenic O-antigen variants of Salmonella typhi. Mol. Microbiol. 1994, 13(3), 525-530.
5. Noriega, F. R., Wang, J. Y., Losonsiy, G., Maneval, D. R., Hone, D. M. & Levine, M. M. Construction and characterization of attenuated delta aroA delta virG Shigella flexneri 2a strain CVD 1203, a prototype live oral vaccine. Infect. Immun. 1994, 62(11), 5168-5172.
6. Fouts, T. R., Tuskan, R. G., Chada, S., Hone, D. M. & Lewis, G. K. Construction and immunogenicity of Salmonella typhimurium vaccine vectors that express HIV-1 gp120. Vaccine 1995, 13(17), 1697-1705.
7. Fouts, T. R., Lewis, G. K. & Hone, D. M. Construction and characterization of a Salmonella typhi-based human immunodeficiency virus type 1 vector vaccine. Vaccine 1995, 13(6), 561-569.
8. Wu, S., Pascual, D. W., VanCott, J. L. et al. Immune responses to novel Escherichia coli and Salmonella typhimurium vectors that express colonization factor antigen I (CFA/I) of enterotoxigenic E. coli in the absence of the CFA/I positive regulator cfaR. Infect. Immun. 1995, 63(12), 4933-4938.
9. Hone, D. M., Wu, S., Powell, R. J. et al. Optimization of live oral Salmonella-HIV-1 vaccine vectors for the induction of HIV-specific mucosal and systemic immune responses, J. Biotech. 1996, 44(1-3), 203-207.
10. Powell, R. J., Lewis, G. K. & Hone, D. M. Introduction of eukaryotic expression cassettes into animal cells using a bacterial vector delivery system. In: Vaccine96. Molecular Approaches to the Control of Infectious Disease. Brown, F., Norrby, E., Burton, D. and Mekalanos, J. (Eds). Cold Spring Harbor Press, New York, N.Y. 1996, Pp 183-187.
11. Wu, S., Pascual, D. W., Lewis, G. K. & Hone, D. M. Induction of mucosal and systemic responses against human immunodeficiency virus type 1 glycoprotein 120 in mice after oral immunization with a single dose of a Salmonella-HIV vector. AIDS Res. Hum. Retrovir. 1997, 13(14), 1187-1194.
12. Ansorge, W., Sproat, B., Stegemann, J., Schwager, C. & Zenke, M. Automated DNA sequencing: ultrasensitive detection of fluorescent bands during electrophoresis. Nucleic Acids Res 1987, 15(11), 4593-4602.
13. Bagley, K. C., Fouts, T. R., Carbonetti, N., DeVico, A. L., Lewis, G. K. & Hone, D. M. Immunogenicity of a dicistronic DNA vaccine that directs coincident expression of the 120 kDa glycoprotein of human immunodeficiency virus and the catalytic domain of cholera toxin. (Submitted). 2002.
14. Fouts, T. R., DeVico, A. L., Onyabe, D. Y. et al. Progress toward the development of a bacterial vaccine vector that induces high-titer long-lived broadly neutralizing antibodies against HIV-1. FEMS 2002, (In press).
15. Giuliani, M. M., Del Giudice, G., Giannelli, V. et al. Mucosal adjuvanticity and immunogenicity of LTR72, a novel mutant of Escherichia coli heat-labile enterotoxin with partial knockout of ADP-ribosyltransferase activity. J. Exp. Med. 1998, 187(7), 1123-1132.
16. Porgador, A., Irvine, K. R., Iwasaki, A., Barber, B. H., Restifo, N. P. & Germain, R. N. Predominant role for directly transfected dendritic cells in antigen presentation to CD8+ T cells after gene gun immunization. J. Exp. Med. 1998, 188(6), 1075-1082.
17. Casares, S., Inaba, K., Brumeanu, T. D., Steinman, R. M. & Bona, C. A. Antigen presentation by dendritic cells after immunization with DNA encoding a major histocompatibility complex class II-restricted viral epitope. J. Exp. Med. 1997, 186(9), 1481-1486.
18. You, Z., Huang, X., Hester, J., Toh, H. C. & Chen, S. Y. Targeting dendritic cells to enhance DNA vaccine potency. Cancer Res. 2001, 61(9), 3704-3711.

1. A DNA vaccine comprising a nucleotide sequence encoding for an antigen and an ADP-ribosyltransferase toxin devoid of ADP-ribosyltransferase activity.

2. The DNA vaccine according to claim 1, wherein the ADP-ribosyltransferase toxin devoid of ADP-ribosyltransferase activity retains adjuvanticity.

3. The DNA vaccine according to claim 1, wherein the ADP-ribosyltransferase toxin devoid of ADP-ribosyltransferase activity comprises a cholera toxin.

4. The DNA vaccine according to claim 1, wherein the ADP-ribosyltransferase toxin devoid of ADP-ribosyltransferase activity comprises a heat-labile toxin.

5. The DNA vaccine according to claim 1, wherein the ADP-ribosyltransferase toxin devoid of ADP-ribosyltransferase activity comprises SEQ ID NO. 5 or nucleotide variant thereof.

6. The DNA vaccine according to claim 1, wherein the antigen comprises a viral, bacterial, parasitic, autoimmune or transplantation antigen.

7. The DNA vaccine according to claim 1, wherein the ADP-ribosyltransferase toxin devoid of ADP-ribosyltransferase activity comprises a mutated A1 domain of the A subunit of CT to inhibit ADP-ribosyltransferase activity

8. The DNA vaccine according to claim 1, wherein the ADP-ribosyltransferase toxin devoid of ADP-ribosyltransferase activity comprises a mutation in cholera toxin selected from the group consisting of R7K, S61K, S63K, V53D, V97K, Y104K and combinations thereof.

9. The DNA vaccine according to claim 1, wherein the ADP-ribosyltransferase toxin devoid of ADP-ribosyltransferase activity comprises a mutation in a A subunit of heat-labile toxin selected from the group consisting of R7K, S61K, S63K, V53D, V97K, Y104K, and combinations thereof.

10. The DNA vaccine according to claim 1, wherein the antigen is gp120.

11. The DNA vaccine according to claim 1, wherein the nucleotide sequence encoding an ADP-ribosyltransferase toxin devoid of ADP-ribosyltransferase activity is in a plasmid or expression vector.

12. The DNA vaccine according to claim 11, wherein is dicistronic and further comprises an antigen operably linked to transcription regulatory elements.

13. The DNA Vaccine according to claim 11, further comprises an antigen operably linked to transcription regulatory elements.

14. The DNA vaccine according to claim 13, wherein the ADP-ribosyltransferase toxin devoid of ADP-ribosyltransferase activity retains adjuvanticity.

15. The DNA vaccine according to claim 13, wherein the ADP-ribosyltransferase toxin devoid of ADP-ribosyltransferase activity comprises a cholera toxin.

16. The DNA vaccine according to claim 13, wherein the ADP-ribosyltransferase toxin devoid of ADP-ribosyltransferase activity comprises a heat-labile toxin.

17. The DNA vaccine according to claim 13, wherein the ADP-ribosyltransferase toxin devoid of ADP-ribosyltransferase activity comprises SEQ ID NO. 5 or nucleotide variant thereof.

18. The DNA vaccine according to claim 13, wherein the antigen comprises a viral, bacterial, parasitic, autoimmune or transplantation antigen.

19. The DNA vaccine according to claim 13, wherein the ADP-ribosyltransferase toxin devoid of ADP-ribosyltransferase activity comprises a mutated A1 domain of the A subunit of CT to inhibit ADP-ribosyltransferase activity

20. The DNA vaccine according to claim 13, wherein the ADP-ribosyltransferase toxin devoid of ADP-ribosyltransferase activity comprises a mutation in cholera toxin selected from the group consisting of R7K, S61K, S63K, V53D, V97K, Y104K and combinations thereof.

21. The DNA vaccine according to claim 13, wherein the ADP-ribosyltransferase toxin devoid of ADP-ribosyltransferase activity comprises a mutation in a A subunit of heat-labile toxin selected from the group consisting of R7K, S61K, S63K, V53D, V97K, Y104K, and combinations thereof.

22. The DNA vaccine according to claim 13, wherein the antigen is gp120.

23. The DNA vaccine according to claim 11, wherein the nucleotide sequence encoding for an antigen and an ADP-ribosyltransferase toxin devoid of ADP-ribosyltransferase activity is operably linked to transcription regulatory.

24.-31. (canceled)

32. A recombinant host cell transfected with the plasmid or expression vector of claim 11.

33. A method of vaccination comprising:
administering a DNA vaccine comprising a nucleotide sequence encoding for an antigen and an adjuvant, wherein the adjuvant is an ADP-ribosyltransferase toxin devoid of ADP-ribosyltransferase activity.

34. The method of vaccination according to claim 33, wherein the ADP-ribosyltransferase toxin devoid of ADP-ribosyltransferase activity is a mutated cholera toxin.

35. The method of vaccination according to claim 34, wherein the mutated cholera toxin comprises CtxA1-K63.

36. The method of vaccination according to claim 33, wherein the antigen comprises gp120.

37. The method of vaccination according to claim 33, wherein the antigen is derived from a member selected from the group consisting of Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

38. A method of inducing an immune response, comprising administering the DNA vaccine of according to claim 1, wherein the ADP-ribosyltransferase toxin devoid of ADP-ribosyltransferase activity comprises a mutated cholera toxin or a mutated heat labile toxin.

39.-40. (canceled)

41. A composition comprising a nucleotide sequence encoding for an antigen and an ADP-ribosyltransferase toxin devoid of ADP-ribosyltransferase activity according to claim 1.

42.-49. (canceled)

50. The composition according to claim 41, wherein the antigen is gp120.

51. The composition according to claim 41, wherein the antigen is derived from a member selected from the group consisting of Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.


SEQUENCE LISTING (7 sequences)

SEQ ID NO: 1 - DNA, 39 bp, Artificial Sequence
  Synthetic Construct
ggggggggatccatgcccatggggtctctgcaaccgctg

SEQ ID NO: 2 - DNA, 45 bp, Artificial Sequence
  Synthetic Construct
gggggcggccgcttattaggcgcgcttctcgcgctgcaccacgcg

SEQ ID NO: 3 - DNA, 71 bp, Artificial Sequence
  Synthetic Construct
ataagaatgcggccgctaagtaagtaacttaagttccggttattttccacgatattgccgtcttttggcaa

SEQ ID NO: 4 - DNA, 42 bp, Artificial Sequence
  Synthetic Construct
gccaaatacatggccatattatcatcgtgtttttcaaaggaa

SEQ ID NO: 5 - DNA, 582 bp, Artificial Sequence
  Synthetic Construct
aatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccaccaaaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcg

SEQ ID NO: 6 - DNA, 31 bp, Artificial Sequence
  Synthetic Construct
tgtttcccaccaaaattagtttgagaagtgc

SEQ ID NO: 7 - DNA, 30 bp, Artificial Sequence
  Synthetic Construct
caaactaattttggtggaaacatatccatc